(19) United States
(12) Patent Application Publication
(10) Pub. No.: US 2024/0352518 A1
(43) Pub. Date: Oct. 24, 2024

(54) METHODS FOR SIMULTANEOUS MUTATION DETECTION AND METHYLATION ANALYSIS

(71) Applicant: The Johns Hopkins University, Baltimore, MD (US)

(72) Inventors: Bert Vogelstein, Baltimore, MD (US); Kenneth W. Kinzler, Frankford, DE (US); Nickolas Papadopoulos, Towson, MD (US); Austin Mattox, Baltimore, MD (US); Joshua David Cohen, Baltimore, MD (US); Yuxuan Wang, Baltimore, MD (US)

(21) Appl. No.: 18/682,716

(22) PCT Filed: Aug. 12, 2022
(86) PCT No.: PCT/US22/40174

§ 371 Date: Feb. 9, 2024

Related U.S. Application Data
(60) Provisional application No. 63/232,438, filed on Aug. 12, 2021.

(51) Int. Cl. C12Q 1/6874; C12Q 1/6806; C12Q 1/6844

(52) U.S. Cl. CPC C12Q 1/6874; C12Q 1/6844; C12Q 1/6806; C12Q 2600/154

(57) ABSTRACT

Provided herein are methods for identifying a genetic characteristic, a fragment characteristic and an epigenetic characteristic of a double-stranded DNA molecule in a population of double-stranded DNA molecules by assaying both strands of the double-stranded DNA molecule.

### CROSS-REFERENCE TO RELATED APPLICATIONS

This application claims priority to U.S. Provisional Patent Application No. 63/232,438, filed on Aug. 12, 2021. The disclosure of this prior application is considered part of the disclosure of this application, and is incorporated in its entirety into this application.

### FEDERALLY SPONSORED RESEARCH OR DEVELOPMENT

This invention was made with government support under grant GM136577, GM008752, and CA006973 awarded by the National Institutes of Health. The government has certain rights in the invention.

### TECHNICAL FIELD

The present disclosure relates to the area of nucleic acid analysis. In particular, it relates to nucleic acid sequence analysis which can detect mutations and methylation of the nucleic acid sequence.

### BACKGROUND

The identification of rare mutations is useful in aspects of fundamental biology as well as to improve the clinical management of patients. Fields of use include infectious diseases, immune repertoire profiling, palentogenetics, forensics, aging, non-invasive prenatal testing, and cancer. Next generation sequencing (NGS) technologies are theoretically suitable for this application, and a variety of NGS approaches exist for the detection of rare mutations. However, for conventional NGS approaches, the error rate of the sequencing itself is too high to allow confident detection of mutations, particularly those mutations present at low frequencies in the original sample.

The use of molecular barcodes to tag original template molecules was designed to overcome various obstacles in the detection of rare mutations. With molecular barcoding, redundant sequencing of the PCR-generated progeny of each tagged molecule is performed and sequencing errors are easily recognized. For example, if a given threshold of the progeny of the barcoded template molecule contain the same mutation, then the mutation is considered genuine. If less than a given threshold of the progeny contain the mutation of interest, then the mutation is considered an artifact. Two types of molecular barcodes have been described: exogenous and endogenous. Exogenous barcodes (also referred to as exogenous unique identifiers, or “UIDs”) comprise pre-specified or random nucleotides, and are appended during library preparation or during PCR. Endogenous barcodes (also referred to as endogenous UIDs) are formed by the sequences present in the template DNA to be assayed, e.g., fragments generated by random shearing of DNA or fragments present in a cell-free fluid biological sample. In some cases, endogenous barcodes are sequences present at the 5′ and/or 3′ ends of fragments. Such barcodes have been proven useful for tracing amplicons back to an original starting template, allowing for molecular counting and improving the identification of true mutations in clinically-relevant samples.

The identification of rare epigenetic changes in DNA, such as those associated with methylation or hydroxymethylation of cytosines, presents similar challenges to those described above for mutations. At present, experimental techniques to evaluate mutations at very high specificity in plasma (for example) requires one aliquot of plasma. Experimental techniques to evaluate epigenetic changes require a second aliquot of plasma. Because of the rarity of certain mutations and epigenetic changes in plasmas from early stage cancer patients, it would be advantageous to evaluate as many molecules as possible for both genetic and epigenetic changes. Splitting a sample—half for genetic changes, half for epigenetic changes—reduces sensitivity by 50% for both types of alterations.

Accordingly, there exists a need for improvements to sequencing library preparation and workflow, to enable accurate identification of mutations, e.g., rare mutations, as well as epigenetic changes, from the same aliquot of DNA purified from clinically relevant samples such as, without limitation, plasma.

### SUMMARY

Provided herein are methods for identifying a genetic characteristic and an epigenetic characteristic of a double-stranded DNA molecule in a population of double-stranded DNA molecules by assaying at least one strand of the double-stranded DNA molecule, the method comprising: (a) attaching an adapter fragment to each end of the double-stranded DNA molecule to generate an adapted double-stranded DNA molecule, wherein the adapted double-stranded DNA molecule comprises an adapted Watson strand and an adapted Crick strand, wherein the adapter fragment comprises a molecular barcode, a primer sequence, and an adapter sequence, and wherein the molecular barcode of the adapted Watson strand is the reverse complement of the molecular barcode of the adapted Crick strand; (b) copying both strands of the adapted double-stranded DNA molecule, wherein the copying comprises (i) contacting the adapted double-stranded DNA molecule with a tagged primer and (ii) performing a round of linear extension of the adapted double-stranded DNA molecule, generating a tagged Watson strand and a tagged Crick strand; (c) subjecting the amplified products to denaturing conditions; (d) separately recovering the adapted Watson and Crick strands and the tagged Watson and Crick strands; (e) generating a first population of analyte DNA fragments from the tagged Watson and Crick strands and generating a first sequencing read for at least one member of the first population of analyte DNA fragments; (f) generating a second population of analyte DNA fragments from the adapted Watson and Crick strands and generating a second sequencing read for at least one member of the second population of analyte DNA fragments; (g) grouping the first sequencing reads according to the molecular barcode present on the at least one member of the first population of analyte DNA fragments to generate a first analyte DNA family; (h) grouping the second sequencing reads according to the molecular barcode present on the at least one member of the second population of analyte DNA fragments to generate a second analyte DNA family; (i) identifying the genetic characteristic of the tagged Watson and Crick strands in the first analyte DNA family; and (j) identifying the epigenetic characteristic of the adapted Watson and Crick strands in the second analyte DNA family, thus, identifying the genetic characteristic and the epigenetic characteristic present on at least one strand of the double stranded DNA molecule.

In some embodiments, the adaptor fragment further comprises a sample barcode. In some embodiments, the molecular barcode comprises an endogenous barcode, an exogenous barcode, or both.

In some embodiments, the copying step (b) comprises performing one, two, or three round(s) of linear extension of the adapted double-stranded DNA molecule. In some embodiments, the tagged primer is a uracil-containing biotinylated primer, and wherein the tagged Watson and Crick strands are generated from the uracil-containing biotinylated primer.

In some embodiments, the recovering step (d) comprises contacting the tagged Watson and Crick strands with streptavidin-functionalized beads, and wherein the tagged Watson and Crick strands bind the streptavidin-functionalized beads. In some embodiments, the recovered adapted Watson and Crick strands that are not bound to the streptavidin-functionalized beads are treated with bisulfite to convert Cytosine bases to Uracil bases to generate the second population of analyte DNA fragments comprising a population of converted DNA molecules.

In some embodiments, the denaturing conditions comprise NaOH denaturation. In some embodiments, the denaturing conditions comprise heat denaturation, chemical denaturation, or combinations thereof. In some embodiments, the generating steps (e) and (f) are performed under PCR conditions.

In some embodiments, the genetic characteristic is a mutation. In some embodiments, the mutation is selected from the group consisting of an insertion, a deletion, a substitution, a deletion-insertion, a duplication, an inversion, a frameshift, a repeat expansion, a translocation, and combinations thereof. In some embodiments, the epigenetic characteristic is methylation. In some embodiments, the epigenetic characteristic is a methylation pattern. In some embodiments, the methylation pattern corresponds to a methylation pattern present in cells generated via clonal hematopoiesis of indeterminate origin. In some embodiments, the methylation pattern corresponds to a methylation pattern present in a tissue of origin. In some embodiments, the tissue of origin is the anus, bladder/urothelial, breast, cervix, colon/rectum, head and neck, kidney, liver/bile duct, lung, lymphoid neoplasm, melanoma, myeloid neoplasm, ovary, pancreas/gallbladder, prostate, thyroid, upper GI, or uterus. In some embodiments, the epigenetic characteristic is hydroxymethylation, histone modification, microRNA regulation, acetylation, phosphorylation, ubiquitination, or sumoylation. In some embodiments, the method identifies a genetic characteristic and an epigenetic characteristic of a double-stranded DNA molecule in a population of double-stranded DNA molecules by assaying both strands of the double-stranded DNA molecule.

Also provided herein are methods for identifying a first characteristic and a second characteristic of a double stranded DNA molecule in a population of double-stranded DNA molecules by assaying at least one strand of the double-stranded DNA molecule, the method comprising: (a) attaching an adapter fragment to each end of the double-stranded DNA molecule to generate an adapted double-stranded DNA molecule, wherein the adapted double-stranded DNA molecule comprises an adapted Watson strand and an adapted Crick strand, wherein the adapter fragment comprises a molecular barcode, a primer sequence, and an adapter sequence, and wherein the molecular barcode of the adapted Watson strand is the reverse complement of the molecular barcode of the adapted Crick strand; (b) copying both strands of the adapted double-stranded DNA molecule, wherein the copying comprises (i) contacting the adapted double-stranded DNA molecule with a tagged primer and (ii) performing a round of linear extension of the adapted double-stranded DNA molecule, generating a tagged Watson strand and a tagged Crick strand; (c) subjecting the amplified products to denaturing conditions; (d) separately recovering the adapted Watson and Crick strands and the tagged Watson and Crick strands; (e) generating a first population of analyte DNA fragments from the tagged Watson and Crick strands and generating a first sequencing read for at least one member of the first population of analyte DNA fragments; (f) generating a second population of analyte DNA fragments from the adapted Watson and Crick strands and generating a second sequencing read for at least one member of the second population of analyte DNA fragments; (g) grouping the first sequencing reads according to the molecular barcode present on the at least one member of the first population of analyte DNA fragments to generate a first analyte DNA family; (h) grouping the second sequencing reads according to the molecular barcode present on the at least one member of the second population of analyte DNA fragments to generate a second analyte DNA family; (i) identifying the first characteristic of the tagged Watson and Crick strands in the first analyte DNA family; and (j) identifying the second characteristic of the adapted Watson and Crick strands in the second analyte DNA family, thus, identifying the first characteristic and the second characteristic present on at least one strand of the double-stranded DNA molecule.

In some embodiments, the adaptor fragment further comprises a sample barcode. In some embodiments, the molecular barcode comprises an endogenous barcode, an exogenous barcode, or both.

In some embodiments, the copying step (b) comprises performing one, two, or three round(s) of linear extension of the adapted double-stranded DNA molecule. In some embodiments, the tagged primer is a uracil-containing biotinylated primer, and wherein the tagged Watson and Crick strands are generated from the uracil-containing biotinylated primer.

In some embodiments, the recovering step (d) comprises contacting the first single stranded DNA fragment with streptavidin-functionalized beads, and wherein the first single-stranded DNA fragment binds the streptavidin-functionalized beads. In some embodiments, the denaturing conditions comprise NaOH denaturation. In some embodiments, the denaturing conditions comprise heat denaturation, chemical denaturation, or combinations thereof.

In some embodiments, the generating steps (e) and (f) are performed under PCR conditions. In some embodiments, the generating employs whole-genome PCR, whole-genome bisulfite sequencing, or capture sequencing.

In some embodiments, the first characteristic is a genetic characteristic or an epigenetic characteristic. In some embodiments, the second characteristic is an epigenetic characteristic or an epigenetic characteristic. In some embodiments, the first characteristic and second characteristic are both genetic characteristics. In some embodiments, the first characteristic and second characteristic are both epigenetic characteristic.

In some embodiments, the genetic characteristic is a mutation. In some embodiments, the mutation is selected from the group consisting of an insertion, a deletion, a substitution, a deletion-insertion, a duplication, an inversion, a frameshift, a repeat expansion, a translocation, and combinations thereof. In some embodiments, identifying the genetic characteristic comprises mutational analysis, aneuploidy analysis, or fragmentomics.

In some embodiments, the epigenetic characteristic is methylation. In some embodiments, the epigenetic characteristic is a methylation pattern. In some embodiments, the methylation pattern corresponds to a methylation pattern present in cells generated via clonal hematopoiesis of indeterminate origin. In some embodiments, the methylation pattern corresponds to a methylation pattern present in a tissue of origin. In some embodiments, the tissue of origin is the anus, bladder/urothelial, breast, cervix, colon/rectum, head and neck, kidney, liver/bile duct, lung, lymphoid neoplasm, melanoma, myeloid neoplasm, ovary, pancreas/gallbladder, prostate, thyroid, upper GI, or uterus. In some embodiments, the epigenetic characteristic is hydroxymethylation, histone modification, microRNA regulation, acetylation, phosphorylation, ubiquitination, or sumoylation.

In some embodiments, the method identifies a first characteristic and a second characteristic of a double stranded DNA molecule in a population of double-stranded DNA molecules by assaying both strands of the double-stranded DNA molecule.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows an exemplary workflow for simultaneous mutation detection and methylation analysis.

FIG. 2 shows duplex recovery following workflow described herein.

FIG. 3 shows an exemplary workflow for simultaneous mutation detection and methylation analysis.

FIG. 4 shows an exemplary workflow for simultaneous assessment of somatic mutations and methylation patterns.

FIG. 5 shows an exemplary workflow for mutation analysis and simultaneous mutation and methylation analysis.

### DETAILED DESCRIPTION

The identification of rare mutations or rare epigenetic changes in DNA (e.g., methylation, hydroxymethylation of cytosines) is useful in aspects of fundamental biology as well as to improve the clinical management of patients. At present, conventional techniques to evaluate mutations at very high specificity in plasma require one aliquot of plasma, while conventional techniques to evaluate epigenetic changes require a second aliquot of plasma. Because of the rarity of certain mutations and epigenetic changes in plasmas from early stage cancer patients, it would be advantageous to evaluate as many molecules as possible for both genetic and epigenetic changes. Splitting a sample—half for genetic changes, half for epigenetic changes—reduces sensitivity by 50% for both types of alterations.

Accordingly, there exists a need for improvements to sequencing library preparation and workflow, to enable accurate identification of mutations, e.g., rare mutations, as well as epigenetic changes, from the same aliquot of DNA purified from clinically relevant samples.

Provided herein are methods for identifying a genetic characteristic and an epigenetic characteristic of a double-stranded DNA molecule in a population of double-stranded DNA molecules by assaying at least one strand of the double-stranded DNA molecule, the method including (a) attaching an adapter fragment to each end of the double-stranded DNA molecule to generate an adapted double-stranded DNA molecule, wherein the adapted double-stranded DNA molecule comprises an adapted Watson strand and an adapted Crick strand, wherein the adapter fragment comprises a molecular barcode, a primer sequence, and an adapter sequence, and wherein the molecular barcode of the adapted Watson strand is the reverse complement of the molecular barcode of the adapted Crick strand; (b) copying both strands of the adapted double-stranded DNA molecule, wherein the copying comprises (i) contacting the adapted double-stranded DNA molecule with a tagged primer and (ii) performing a round of linear extension of the adapted double-stranded DNA molecule, generating a tagged Watson strand and a tagged Crick strand; (c) subjecting the amplified products to denaturing conditions; (d) separately recovering the adapted Watson and Crick strands and the tagged Watson and Crick strands; (e) generating a first population of analyte DNA fragments from the tagged Watson and Crick strands and generating a first sequencing read for at least one member of the first population of analyte DNA fragments; (f) generating a second population of analyte DNA fragments from the adapted Watson and Crick strands and generating a second sequencing read for at least one member of the second population of analyte DNA fragments; (g) grouping the first sequencing reads according to the molecular barcode present on the at least one member of the first population of analyte DNA fragments to generate a first analyte DNA family; (h) grouping the second sequencing reads according to the molecular barcode present on the at least one member of the second population of analyte DNA fragments to generate a second analyte DNA family; (i) identifying the genetic characteristic of the tagged Watson and Crick strands in the first analyte DNA family; and (j) identifying the epigenetic characteristic of the adapted Watson and Crick strands in the second analyte DNA family, thus, identifying the genetic characteristic and the epigenetic characteristic present on at least one strand of the double stranded DNA molecule.

Various non-limiting aspects of these methods are described herein, and can be used in any combination without limitation. Additional aspects of various components of methods for identifying the presence or absence of a mutation and methylation are known in the art.

It must be noted that, as used in the specification and the appended claims, the singular forms “a,” “an” and “the” include plural referents unless the context clearly dictates otherwise.

As used herein, an “adaptor,” an “adapter,” and a “tag” are terms that are used interchangeably, and refer to species that can be coupled to a polynucleotide sequence (e.g., in a process referred to as “tagging”) using any one of many different techniques including, but not limited to, ligation, hybridization, and tagmentation. In some embodiments, adaptors can also be nucleic acid sequences that add a function, e.g., spacer sequences, primer sequences/sites, barcode sequences, or unique molecular identifier sequences.

As used herein, the term “barcode” refers to a label, or identifier, that conveys or is capable of conveying information (e.g., information about an analyte in a sample). A barcode can be part of an analyte, or independent of an analyte. In some embodiments, a barcode can be attached to an analyte. In some embodiments, a particular barcode can be unique relative to other barcodes. In some embodiments, barcodes can have a variety of different formats. For example, barcodes can include non-random, semi-random, and/or random nucleic acid and/or amino acid sequences, and synthetic nucleic acid and/or amino acid sequences. In some embodiments, a barcode can be attached to an analyte or to another moiety or structure in a reversible or irreversible manner. In some embodiments, a barcode can be added to, for example, a fragment of a deoxyribonucleic acid (DNA) or ribonucleic acid (RNA) sample before or during sequencing of the sample. In some embodiments, barcodes can allow for identification and/or quantification of individual sequencing-reads. In some embodiments, a barcode can refer to a unique identifier (UID) and the terms “barcode” and “UID” can be used interchangeably.

As used herein, the term “nucleotides” and “nt” are used interchangeably herein to generally refer to biological molecules that comprise nucleic acids. Nucleotides can have moieties that contain the known purine and pyrimidine bases. Nucleotides may have other heterocyclic bases that have been modified. Such modifications include, e.g., methylated purines or pyrimidines, acylated purines or pyrimidines, alkylated riboses, or other heterocycles. The terms “polynucleotides,” “nucleic acid,” and “oligonucleotides” can be used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise non-naturally occurring sequences. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, a “primer” generally refers to a polynucleotide molecule comprising a nucleotide sequence (e.g., an oligonucleotide), generally with a free 3′-OH group, that hybridizes with a template sequence (such as a target polynucleotide, or a primer extension product) and is capable of promoting polymerization of a polynucleotide complementary to the template. In some embodiments, a primer is a biotinylated primer.

### Overview

Provided herein are methods and materials useful for accurately identifying a genetic characteristic and an epigenetic characteristic present in a nucleic acid sample. In some embodiments, the method comprises identifying the genetic and epigenetic characteristics when it is present on at least one of Watson and Crick strands of a double stranded nucleic acid template. In some embodiments, the method comprises identifying the genetic and epigenetic characteristics when it is present on both Watson and Crick strands of a double stranded nucleic acid template. In some embodiments, the double stranded nucleic acid template can include a Watson strand and a Crick strand. In some embodiments, the double stranded nucleic acid template can include a plus strand and a minus strand. In some embodiments, the double stranded nucleic acid template can include a first strand and a second strand. As will be recognized in the art, Watson/Crick, plus/minus, and first/second refer to the two strands of a double stranded nucleic acid molecule. Such methods are particularly useful for distinguishing true mutations from artifacts stemming from, e.g., DNA damage, PCR, and other sequencing artifacts, allowing for the identification of mutations with high confidence.

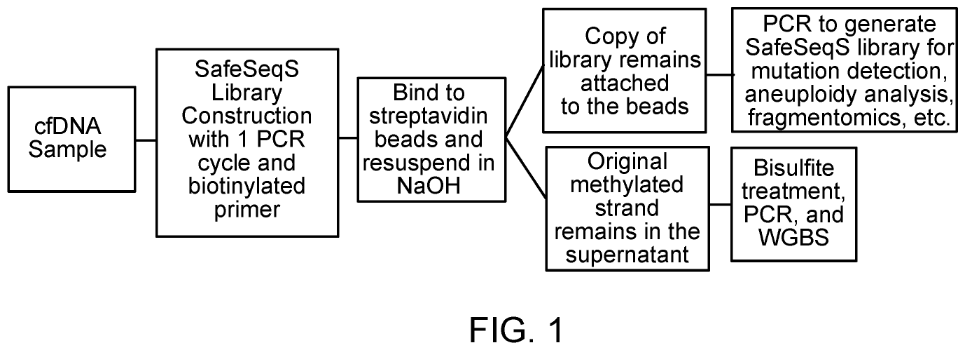

In some embodiments, a method for identifying a genetic characteristic and an epigenetic characteristic of a double-stranded DNA molecule in a population of double-stranded DNA molecules by assaying at least one strand of the double-stranded DNA molecule can include: (a) attaching an adapter fragment to each end of the double-stranded DNA molecule to generate an adapted double-stranded DNA molecule, wherein the adapted double-stranded DNA molecule comprises an adapted Watson strand and an adapted Crick strand, wherein the adapter fragment comprises a molecular barcode, a primer sequence, and an adapter sequence, and wherein the molecular barcode of the adapted Watson strand is the reverse complement of the molecular barcode of the adapted Crick strand; (b) copying both strands of the adapted double-stranded DNA molecule, wherein the copying comprises (i) contacting the adapted double-stranded DNA molecule with a tagged primer and (ii) performing a round of linear extension of the adapted double-stranded DNA molecule, generating a tagged Watson strand and a tagged Crick strand; (c) subjecting the amplified products to denaturing conditions; (d) separately recovering the adapted Watson and Crick strands and the tagged Watson and Crick strands; (e) generating a first population of analyte DNA fragments from the tagged Watson and Crick strands and generating a first sequencing read for at least one member of the first population of analyte DNA fragments; (f) generating a second population of analyte DNA fragments from the adapted Watson and Crick strands and generating a second sequencing read for at least one member of the second population of analyte DNA fragments; (g) grouping the first sequencing reads according to the molecular barcode present on the at least one member of the first population of analyte DNA fragments to generate a first analyte DNA family; (h) grouping the second sequencing reads according to the molecular barcode present on the at least one member of the second population of analyte DNA fragments to generate a second analyte DNA family; (i) identifying the genetic characteristic of the tagged Watson and Crick strands in the first analyte DNA family; and (j) identifying the epigenetic characteristic of the adapted Watson and Crick strands in the second analyte DNA family, thus, identifying the genetic characteristic and the epigenetic characteristic present on at least one strand of the double stranded DNA molecule. In some embodiments, the method comprises identifying the genetic and epigenetic characteristics present on both strands of the double stranded DNA molecule (FIG. 1).

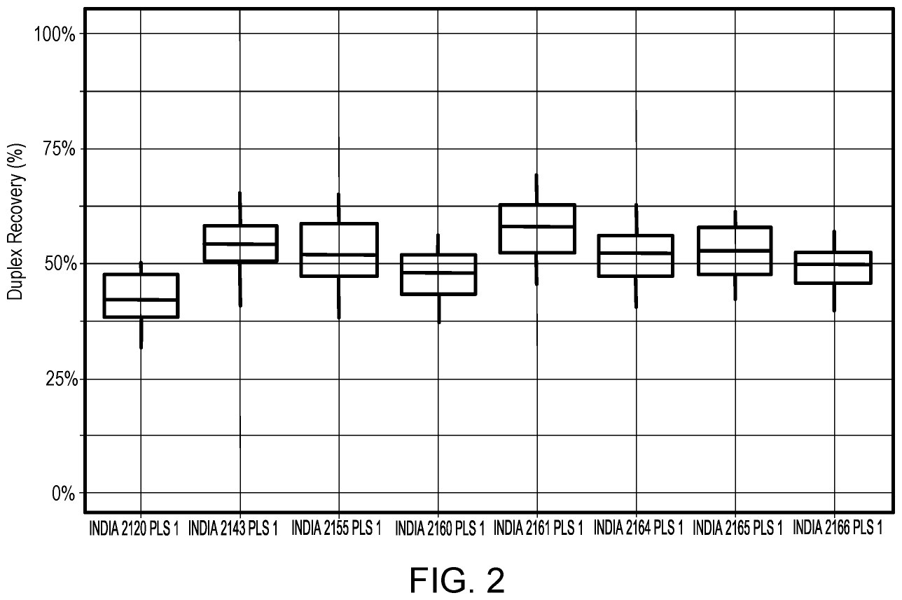

In some cases, the methods and materials described herein can be used to achieve efficient duplex recovery. In some embodiments, methods described herein can be used to recover amplification products derived from at least one of the Watson strand and the Crick strand of a double stranded nucleic acid template. For example, methods described herein can be used to recover amplification products derived from both the Watson strand and the Crick strand of a double stranded nucleic acid template. In some cases, the methods described herein can be used to achieve at least 50% (e.g., about 50%, about 60%, about 70%, about 75%, about 80%, about 82%, about 85%, about 88%, about 90%, about 93%, about 95%, about 97%, about 99%, or 100%) duplex recovery (FIG. 2).

In some embodiments, methods for detecting one or more mutations present on at least one strand of a double stranded nucleic acid can include generating a duplex sequencing library having a duplex molecular barcode on each end (e.g., the 5′ end and the 3′ end) of each nucleic acid in the library, generating a library of single stranded Watson strand-derived sequences and a library of single stranded Crick-strand derived sequences from the duplex sequencing library, and detecting the presence of one or more mutations present on at least one strand of the double stranded nucleic acid in each single stranded library. In some embodiments, methods for detecting one or more mutations present on both strands of a double stranded nucleic acid can include generating a duplex sequencing library having a duplex molecular barcode on each end (e.g., the 5′ end and the 3′ end) of each nucleic acid in the library, generating a library of single stranded Watson strand-derived sequences and a library of single stranded Crick-strand derived sequences from the duplex sequencing library, and detecting the presence of one or more mutations present on both strands of the double stranded nucleic acid in each single stranded library. The presence of a first molecular barcode in a 3′ duplex adapter and a second molecular barcode present in a 5′ adapter can be used to distinguish amplification products derived from the Watson strand from amplification products derived from the Crick strand.

In some cases, the methods and materials described herein can be used to independently assess each strand of a double stranded nucleic acid. For example, when a nucleic acid mutation is identified in independently assessed strands of a double stranded nucleic acid as described herein, the materials and methods described herein can used to determine from which strand of the double stranded nucleic acid the nucleic acid mutation originated.

Any appropriate method can be used to generate a duplex sequencing library. As used herein a duplex sequencing library is a plurality of nucleic acid fragments including a duplex molecular barcode on at one end (e.g., the 5′ end and/or the 3′ end) of each nucleic acid fragment in the library and can allow at least one strand of a double stranded nucleic acid to be sequenced. In some embodiments, both strands of the double stranded nucleic acid are sequenced. In some cases, a nucleic acid sample (e.g., double stranded DNA molecule) can be fragmented to generate nucleic acid fragments (e.g., analyte DNA fragments), and the generated nucleic acid fragments can be used to generate a duplex sequencing library. Nucleic acid fragments used to generate a duplex sequencing library can also be referred to herein as input nucleic acid. For example, when nucleic acid fragments used to generate a duplex sequencing library are DNA fragments, the DNA fragments can also be referred to herein as input DNA. A duplex sequencing library can include any appropriate number of nucleic acid fragments. In some cases, generating a duplex sequencing library can include fragmenting a nucleic acid template and ligating adapters to each end of each nucleic acid fragment in the library.

### (1) Adapted Double-Stranded DNA Molecule

In some embodiments, a method described herein can include (a) attaching an adapter fragment to each end of the double-stranded DNA molecule to generate an adapted double-stranded DNA molecule, wherein the adapted double-stranded DNA molecule includes an adapted Watson strand and an adapted Crick strand, wherein the adapter fragment includes a molecular barcode, a primer sequence, and an adapter sequence, and wherein the molecular barcode of the adapted Watson strand is the reverse complement of the molecular barcode of the adapted Crick strand; and (b) copying both strands of the adapted double-stranded DNA molecule, wherein the copying includes (i) contacting the adapted double-stranded DNA molecule with a tagged primer and (ii) performing a round of linear extension of the adapted double-stranded DNA molecule, generating a tagged Watson strand and a tagged Crick strand.

### Analyte Nucleic Acids

Nucleic acids to be analyzed by any of the variety methods provided herein can include any type of nucleic acid (e.g., DNA, RNA, and DNA/RNA hybrids). Examples of nucleic acids that can be analyzed include, but are not limited to, genomic DNA and cell-free DNA (cfDNA) (e.g., circulating tumor DNA (ctDNA), or cell-free fetal DNA (cffDNA). In some embodiments, a nucleic acid to be analyzed can be a double-stranded DNA molecule. In some embodiments, a double-stranded DNA molecule can include a Watson strand, wherein the Watson strand is a first single-strand of the double-stranded DNA molecule. In some embodiments, a double-stranded DNA molecule can include a Crick strand, wherein the Crick strand is a second single-strand of the double-stranded DNA molecule.

In some embodiments, the double-stranded DNA molecules to be analyzed are nucleic acid fragments (e.g., DNA fragment). In some embodiments, the nucleic acid fragments are manually produced. In some embodiments, the fragments are produced by shearing (e.g., enzymatic shearing, shearing by chemical means, acoustic shearing, nebulization, centrifugal shearing, point-sink shearing, needle shearing, sonication, restriction endonucleases, non-specific nucleases (e.g., DNase I), or any combination thereof). In some embodiments, the nucleic acid fragments are naturally produced in the subject. For example, nucleic acid fragments to be analyzed can be cfDNA (e.g., circulating tumor DNA (ctDNA), or cell-free fetal DNA (cffDNA).

In some embodiments, a nucleic acid fragment to be analyzed has a length of about 4 to about 1000 nucleotides (e.g., about 10 to about 1000, about 20 to about 1000, about 30 to about 1000, about 40 to about 1000, about 50 to about 1000, about 60 to about 1000, about 70 to about 1000, about 80 to about 1000, about 90 to about 1000, about 100 to about 1000, about 250 to about 1000, about 500 to about 1000, about 750 to about 1000, about 4 to about 750, about 10 to about 750, about 20 to about 750, about 30 to about 750, about 40 to about 750, about 50 to about 750, about 60 to about 750, about 70 to about 750, about 80 to about 750, about 90 to about 750, about 100 to about 750, about 250 to about 750, about 500 to about 750, about 4 to about 500, about 10 to about 500, about 20 to about 500, about 30 to about 500, about 40 to about 500, about 50 to about 500, about 60 to about 500, about 70 to about 500, about 80 to about 500, about 90 to about 500, about 100 to about 500, about 250 to about 500, about 4 to about 250, about 10 to about 250, about 20 to about 250, about 30 to about 250, about 40 to about 250, about 50 to about 250, about 60 to about 250, about 70 to about 250, about 80 to about 250, about 90 to about 250, about 100 to about 250, about 4 to about 100, about 10 to about 100, about 20 to about 100, about 30 to about 100, about 40 to about 100, about 50 to about 100, about 60 to about 100, about 70 to about 100, about 80 to about 100, about 90 to about 100, about 4 to about 90, about 10 to about 90, about 20 to about 90, about 30 to about 90, about 40 to about 90, about 50 to about 90, about 60 to about 90, about 70 to about 90, about 80 to about 90, about 4 to about 80, about 10 to about 80, about 20 to about 80, about 30 to about 80, about 40 to about 80, about 50 to about 80, about 60 to about 80, about 70 to about 80, about 4 to about 70, about 10 to about 70, about 20 to about 70, about 30 to about 70, about 40 to about 70, about 50 to about 70, about 60 to about 70, about 4 to about 60, about 10 to about 60, about 20 to about 60, about 30 to about 60, about 40 to about 60, about 50 to about 60, about 4 to about 50, about 10 to about 50, about 20 to about 50, about 30 to about 50, about 40 to about 50, about 4 to about 40, about 10 to about 40, about 20 to about 40, about 30 to about 40, about 4 to about 30, about 10 to about 30, about 20 to about 30, about 4 to about 20, about 10 to about 20, or about 4 to about 10). In some embodiments, the length of the nucleic acid fragment to be analyzed may be less than 1000 (e.g., less than 750, less than 500, less than 250, less than 100, less than 50, or less than 20) nucleotides.

In some embodiments, sequences present in nucleic acids to be analyzed (e.g., one or both ends of the nucleic acid) are used as endogenous barcodes. In some embodiments, the ends of a DNA fragment represent unique sequences which can be used as an endogenous barcode (e.g., unique identifier) of the fragment. A skilled artisan may determine the length of the endogenous barcode needed to uniquely identify a nucleic acid template, using factors such as, e.g., overall template length, complexity of nucleic acid templates in a partition or starting nucleic acid sample, and the like. In some embodiments, about 10 to about 500 nucleotides (e.g., about 25 to about 500, about 50 to about 500, about 100 to about 500, about 250 to about 500, about 10 to about 250, about 25 to about 250, about 50 to about 250, about 100 to about 250, about 10 to about 100, about 25 to about 100, about 50 to about 100, about 10 to about 50, about 25 to about 50, or about 10 to about 25 nucleotides) of the ends of nucleic acid templates are used as endogenous barcodes. In some embodiments, both ends of a nucleic acid template are used as an endogenous barcode. In some embodiments, only one end of a nucleic acid template is used as an endogenous barcode.

In some embodiments, the nucleic acid to be analyzed is present in and/or can be obtained from a biological sample. The biological sample may be obtained from a subject. In some embodiments, the subject is a mammal. Examples of mammals from which nucleic acid can be obtained and used as a nucleic acid template in the methods described herein include, without limitation, humans, non-human primates (e.g., monkeys), dogs, cats, sheep, rabbits, mice, hamsters, and rats. In some embodiments, the subject is a human subject.

Biological samples include, but are not limited to, plasma, serum, blood, tissue, tumor sample, stool, sputum, saliva, urine, sweat, tears, ascites, bronchoaveolar lavage, semen, archeologic specimens, and forensic samples. In some embodiments, the biological sample is a solid biological sample, e.g., a tumor sample. In some embodiments, the solid biological sample is processed. The solid biological sample may be processed by fixation in a formalin solution, followed by embedding in paraffin (e.g., is a FFPE sample). Processing can alternatively comprise freezing of the sample prior to conducting the probe-based assay. In some embodiments, the sample is neither fixed nor frozen. The unfixed, unfrozen sample can be, by way of example only, stored in a storage solution configured for the preservation of nucleic acid.

In some embodiments, the biological sample is a liquid biological sample. Liquid biological samples include, but are not limited to, plasma, serum, blood, sputum, saliva, urine, sweat, tears, ascites, bronchoaveolar lavage, and semen. In some embodiments, the liquid biological sample is cell-free or substantially cell-free. In some embodiments, the biological sample is a plasma or serum sample. In some embodiments, the liquid biological sample is a whole blood sample. In some embodiments, the liquid biological sample includes peripheral mononuclear blood cells.

In some embodiments, a nucleic acid to be analyzed is isolated and purified from the biological sample. Nucleic acids can be isolated and purified from a biological sample using any means known in the art. For example, a biological sample may be processed to release nucleic acids from cells, or to separate nucleic acids from unwanted components of the biological sample (e.g., proteins, cell walls, other contaminants). Additionally or alternatively, nucleic acids can be extracted from the biological sample using liquid extraction (e.g., Trizol, DNAzol) techniques. Nucleic acids can also be extracted using commercially available kits (e.g., Qiagen DNeasy kit, QIAamp kit, Qiagen Midi kit, QIAprep spin kit).

Nucleic acids can be concentrated by known methods, including, by way of example only, centrifugation. Nucleic acids can be bound to a selective membrane (e.g., silica) for the purposes of purification. Nucleic acids can also be enriched for fragments of a desired length, e.g., fragments which are less than 1000, 500, 400, 300, 200 or 100 base pairs in length. Such an enrichment based on size can be performed using, e.g., PEG-induced precipitation, an electrophoretic gel or chromatography material (Huber et al. (1993) Nucleic Acids Res. 21:1061-6), gel filtration chromatography, TSK gel (Kato et al. (1984) J. Biochem, 95:83-86), which publications are hereby incorporated by reference.

In some embodiments, a nucleic acid sample that includes the nucleic acid/s to be analyzed includes less than about 35 ng of nucleic acid. For example, the nucleic acid sample can include from about 1 ng to about 35 ng of nucleic acid (e.g., from about 1 ng to about 30 ng, from about 1 ng to about 25 ng, from about 1 ng to about 20 ng, from about 1 ng to about 15 ng, from about 1 ng to about 10 ng, from about 1 ng to about 5 ng, from about 5 ng to about 35 ng, from about 5 ng to about 30 ng, from about 5 ng to about 25 ng, from about 5 ng to about 20 ng, from about 5 ng to about 15 ng, from about 5 ng to about 10 ng, from about 10 ng to about 35 ng, from about 10 ng to about 30 ng, from about 10 ng to about 25 ng, from about 10 ng to about 20 ng, from about 10 ng to about 25 ng, from about 10 ng to about 20 ng, from about 10 ng to about 15 ng, from about 15 ng to about 35 ng, from about 15 ng to about 30 ng, from about 15 ng to about 25 ng, from about 15 ng to about 20 ng, from about 20 ng to about 35 ng, from about 20 ng to about 30 ng, from about 20 ng to about 25 ng, from about 25 ng to about 35 ng, from about 25 ng to about 30 ng, or from about 30 ng to about 35 ng of nucleic acid). In some cases, a nucleic acid sample can include nucleic acid/s from a genome that includes more than about several hundred nucleotides of nucleic acid.

In some cases, a nucleic acid sample that includes the nucleic acid/s to be analyzed can be essentially free of contamination. For example, when a nucleic acid sample includes a cfDNA nucleic acid to be analyzed, the cfDNA can be essentially free of genomic DNA contamination. In some cases, a nucleic acid sample that includes cfDNA that is essentially free of genomic DNA contamination can include minimal (or no) high molecular weight (e.g., >1000 bp) DNA. In some cases, methods described herein can include determining whether a nucleic acid sample is essentially free of contamination. Any appropriate method can be used to determine whether a nucleic acid sample is essentially free of contamination. Examples of methods that can be used to determine whether a nucleic acid sample is essentially free of contamination include, for example, a TapeStation system, and a Bioanalyzer. For example, when using a TapeStation system and/or a Bioanalyzer to determine whether a cfDNA sample is essentially free of genomic DNA contamination, a prominent peak at ˜180 bp (e.g., corresponding to mononucleosomal DNA) can be used to indicate that the nucleic acid sample is essentially free of genomic DNA contamination.

In some cases, nucleic acid fragments that can be used to generate a duplex sequencing library (e.g., prior to attaching a 3′ duplex adapter to the 3′ ends of the nucleic acid fragments) can be end-repaired. Any appropriate method can be used to end-repair a nucleic acid template. For example, blunting reactions (e.g., blunt end ligations) and/or dephosphorylation reactions can be used to end-repair a nucleic acid template. In some cases, blunting can include filling in a single stranded region. In some cases, blunting can include degrading a single stranded region. In some cases, blunting and dephosphorylation reactions can be used to end-repair a nucleic acid template.

### Adapters

As used herein, an “adapter” and “adapter fragment” can refer to a species that can be coupled to a polynucleotide sequence using any one of many different techniques including, but not limited to, ligation, hybridization, and tagmentation. In some embodiments, adapter fragments can also be nucleic acid sequences that add a function, e.g., spacer sequences, primer sequences/sites, or barcode sequences (e.g., UID sequences).

In some embodiments, methods described herein include attaching an adapter fragment to each end of a double-stranded DNA molecule to generate an adapted double-stranded DNA molecule, wherein the adapted double-stranded DNA molecule comprises an adapted Watson strand and an adapted Crick strand, wherein the adapter fragment comprises a molecular barcode, a primer sequence, and an adapter sequence, and wherein the molecular barcode of the adapted Watson strand is the reverse complement of the molecular barcode of the adapted Crick strand. In some embodiments, the primer sequence can be the reverse complement of the adapter sequence. In some embodiments, the adapter sequence can include specific sequences to allow sequencing when generating a sequence library. In some embodiments, the adapter sequence comprises a sequencing primer sequence (e.g., R1, R2).

In some embodiments, the adapter fragment comprises a double-stranded portion comprising a molecular barcode and a forked portion comprising (i) a single-stranded 3′ adapter sequence and (ii) a single-stranded 5′ adapter sequence. In some embodiments, the single-stranded 3′ adapter sequence is not complementary to the single-stranded 5′ adapter sequence. In some embodiments, the 3′ adapter sequence comprises a second (e.g., R2) sequencing primer site and the 5′ adapter sequence comprises a first (e.g., R1) sequencing primer site. It is to be understood that an “R1” and “R2” sequencing primer sites are used by sequencing systems that produce paired end reads, e.g., reads from opposite ends of a DNA fragment to be sequenced. In some embodiments, the R1 sequencing primer is used to produce a first population of reads from first ends of DNA fragments, and the R2 sequencing primer is used to produce a second population of reads from the opposite ends of the DNA fragments. The first population is referred to herein as “R1” or “Read 1” reads. The second population is referred to herein as “R2” or “Read 2” reads. The R1 and R2 reads can be aligned as “read pairs” or “mate pairs” corresponding to each strand of a double-stranded analyte DNA fragment.

Certain sequencing systems (e.g., Illumina) utilize what they refer to as “R1” and “R2” primers, and “R1” and “R2” reads. It should be noted that the terms “R1” and “R2”, and “Read 1” and “Read 2”, for the purposes of this application, are not limited to how they are referenced in relation to a particular sequencing platform. For example, if an Illumina sequencer is used, the “R2” primer and corresponding R2 read disclosed herein may refer to the Illumina “R2” primer and read, or may refer to the Illumina “R1” primer and read, so long as the “R1” primer and corresponding R1 read disclosed herein refers to the other Illumina primer and read. To clarify, in some embodiments wherein an “R2” primer provided herein is the Illumina “R1” primer producing “R1” reads, the corresponding “R1” primer provided herein is the Illumina “R2” primer producing “R2” reads. To clarify, in some embodiments wherein an “R2” primer provided herein is the Illumina “R2” primer providing “R2” reads, the “R1” primer provided herein is the Illumina “R1” primer providing R1 reads.

In some embodiments, an adapted double-stranded DNA molecule can be a double-stranded DNA molecule wherein an adapter is attached to the double-stranded DNA molecule. In some embodiments, the adapter fragment further includes a sample barcode. In some embodiments, the sample barcode is different from the molecular barcode, wherein the sample barcode is unique to the sample from which the double-stranded DNA molecule was obtained. In some embodiments, a first double-stranded DNA molecule from a first sample can be contacted with a first adapter fragment, wherein the first adapter fragment includes a first sample barcode unique to the first sample. In some embodiments, a second double-stranded DNA molecule from a second sample can be contacted with a second adapter fragment, wherein the second adapter fragment includes a second sample barcode unique to the second sample. In some embodiments, the first adapted double-stranded DNA molecule and the second adapted double-stranded DNA molecule can be mixed in a population of adapted double-stranded DNA molecules, wherein the population of adapted double-stranded DNA molecules are used to in any of the methods described herein. In some embodiments, the mixing of the first and second adapted double-stranded DNA molecules can be performed after the attaching step (a) and the copying step (b). In some embodiments, the mixing of the first and second adapted double-stranded DNA molecules can be performed after contacting the adapted double-stranded DNA molecules with a tagged primer. In some embodiments, the mixing of the first and second adapted double-stranded DNA molecules can be performed after step (c) of subjecting the amplified products to denaturing conditions.

In some embodiments, the population of double-stranded DNA molecules can include a plurality of double-stranded DNA molecules, wherein the plurality of double-stranded DNA molecules include a same sample barcode. In some embodiments, the population of double-stranded DNA molecules can include a plurality of double-stranded DNA molecules, wherein the plurality of double-stranded DNA molecules include different sample barcodes.

### Molecular Barcode

As used herein, “molecular barcode” refers to a barcode that serves to identify individual nucleic acid fragments in an original sample prior to barcoding and amplification. In some embodiments, each individual nucleic acid fragment will have a unique molecular barcode. In some embodiments, barcodes may be randomly generated nucleotide sequences or intentionally chosen nucleotide runs. For attaching molecular barcodes in particular, the number of individual molecular barcodes in a reaction mixture will be in excess of the number of nucleic acid fragments.

In some embodiments, a molecular barcode is unique to each double-stranded DNA fragment in the nucleic acid sample. In some embodiments, the molecular barcode includes an endogenous barcode, an exogenous barcode, or both.

In some embodiments, the molecular barcode has a length of about 2 to about 4000 (e.g., about 2 to about 3500, about 2 to about 3000, about 2 to about 2500, about 2 to about 2000, about 2 to about 1500, about 2 to about 1000, about 2 to about 500, about 2 to about 100, about 2 to about 50, about 2 to about 20, about 2 to about 10, about 10 to about 4000, about 10 to about 3500, about 10 to about 3000, about 10 to about 2500, about 10 to about 2000, about 10 to about 1500, about 10 to about 1000, about 10 to about 500, about 10 to about 100, about 10 to about 50, about 10 to about 20, about 20 to about 4000, about 20 to about 3500, about 20 to about 3000, about 20 to about 2500, about 20 to about 2000, about 20 to about 1500, about 20 to about 1000, about 20 to about 500, about 20 to about 100, about 20 to about 50, about 50 to about 4000, about 50 to about 3500, about 50 to about 3000, about 50 to about 2500, about 50 to about 2000, about 50 to about 1500, about 50 to about 1000, about 50 to about 500, about 50 to about 100, about 100 to about 4000, about 100 to about 3500, about 100 to about 3000, about 100 to about 2500, about 100 to about 2000, about 100 to about 1500, about 100 to about 1000, about 100 to about 500, about 500 to about 4000, about 500 to about 3500, about 500 to about 3000, about 500 to about 2500, about 500 to about 2000, about 500 to about 1500, about 500 to about 1000, about 1000 to about 4000, about 1000 to about 3500, about 1000 to about 3000, about 1000 to about 2500, about 1000 to about 2000, about 1000 to about 1500, about 1500 to about 4000, about 1500 to about 3500, about 1500 to about 3000, about 1500 to about 2500, about 1500 to about 2000, about 2000 to about 4000, about 2000 to about 3500, about 2000 to about 3000, about 2000 to about 2500, about 2500 to about 4000, about 2500 to about 3500, about 2500 to about 3000, about 3000 to about 4000, about 3000 to about 3500, or about 3500 to about 4000) nucleotides. In some embodiments, the length of the molecular barcode is sufficient to uniquely barcode the molecules and the length/sequence of the molecular barcode does not interfere with the downstream amplification steps.

In some embodiments, the molecular barcode sequence can be random. In some embodiments, the molecular barcode sequence can be a random N-mer. For example, if the molecular barcode sequence has a length of six nt, then it may be a random hexamer. If the molecular barcode sequence has a length of 12 nt, then it may be a random 12-mer.

In some embodiments, molecular barcodes can be made using random addition of nucleotides to form a sequence having a length to be used as an identifier. At each position of addition, a selection from one of four deoxyribonucleotides may be used. Alternatively a selection from one of three, two, or one deoxyribonucleotides may be used. Thus the molecular barcode may be fully random, somewhat random, or non-random in certain positions. In some embodiments, the molecular barcodes are not random N-mers, but are selected from a predetermined set of molecular barcode sequences. Exemplary molecular barcodes suitable for use in the methods disclosed herein are described in PCT/US2012/033207, which is hereby incorporated by reference in its entirety.

Attachment of a molecular barcode to a nucleic acid fragment may be performed by any means known in the art, including enzymatic, chemical, or biologic. In some embodiments, one means employs a polymerase chain reaction. In some embodiments, another means employs a ligase enzyme. For example, the ligase enzyme may be mammalian or bacterial. Other enzymes which may be used for attaching are other polymerase enzymes. A molecular barcode may be added to one or both ends of the fragments, preferably to both ends. In some embodiments, a molecular barcode may be contained within a nucleic acid molecule that contains other regions for other intended functionality. For example, a universal priming site may be added to permit later amplification. In some embodiments, another additional site may be a region of complementarity to a particular region or gene in the nucleic acid fragment.

### (2) Tagged Double-Strand DNA Molecule

In some embodiments, a method described herein includes (b) copying both strands of the adapted double-stranded DNA molecule, wherein the copying comprises (i) contacting the adapted double-stranded DNA molecule with a tagged primer and (ii) performing a round of linear extension of the adapted double-stranded DNA molecule, generating a tagged Watson strand and a tagged Crick strand. In some embodiments, the copying step can include performing a single round of linear extension. In some embodiments, the copying step can include performing one, two, or three round(s) of linear extension. In some embodiments, the copying step can include performing one or more rounds (e.g., one, two, three, four, or five) of linear extension. In some embodiments, the tagged primer is a uracil-containing biotinylated primer, and wherein the tagged Watson and Crick strands are generated from the uracil-containing biotinylated primer. In some embodiments, the tagged Watson and Crick strands can be selected using biotinylation-streptavidin affinity in any number of methods known to the field (e.g., streptavidin beads).

As used herein, the term “extension” can refer to a method where two nucleic acid sequences become linked (e.g., hybridized) by an overlap of their respective terminal complementary nucleic acid sequences (i.e., for example, 3′ termini). Such linking can be followed by nucleic acid extension (e.g., an enzymatic extension) of one, or both termini using the other nucleic acid sequence as a template for extension.

In some embodiments, nucleic acid extension generally involves incorporation of one or more nucleic acids (e.g., A, G, C, T, U, nucleotide analogs, or derivatives thereof) into a nucleic acid sequence in a template-dependent manner, such that consecutive nucleic acids are incorporated by an enzyme (such as a polymerase or reverse transcriptase), thereby generating a newly synthesized nucleic acid molecule. In some embodiments, enzymatic extension can be performed by an enzyme including, but not limited to, a polymerase and/or a reverse transcriptase. For example, a primer that hybridizes to a complementary nucleic acid sequence can be used to synthesize a new nucleic acid molecule by using the complementary nucleic acid sequence as a template for nucleic acid synthesis.

In some embodiments, a primer can be a single-stranded nucleic acid sequence having a 3′ end that can be used as a chemical substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). In some embodiments, primers can also include other natural or synthetic nucleotides described herein that can have additional functionality.

In some embodiments, a primer can include a tag, wherein the tag is a molecule or molecular moiety that has a high affinity or preference for associating or binding with another specific or particular molecule or moiety. In some embodiments, the association or binding with another specific or particular molecule or moiety can be via a non-covalent interaction, such as hydrogen bonding, ionic forces, and van der Waals interactions. For example, an affinity group can be biotin which has a high affinity or preference to associate or bind to the protein avidin or streptavidin. Alternatively, an affinity group can also refer to avidin or streptavidin which has an affinity to biotin. Other examples of an affinity group and specific or particular molecule or moiety to which it binds or associates with include, but are not limited to, antibodies or antibody fragments and their respective antigens, such as digoxigenin and anti-digoxigenin antibodies, lectin, and carbohydrates (e.g., a sugar, a monosaccharide, a disaccharide, or a polysaccharide), and receptors and receptor ligands. In some embodiments, the tagged primer is a biotinylated primer, and wherein the tagged Watson and Crick strands are generated from the biotinylated primer. In some embodiments, the tagged primer is a uracil-containing biotinylated primer, and wherein the tagged Watson and Crick strands are generated from the uracil-containing biotinylated primer. In some embodiments, the tagged Watson and Crick strands can be selected using biotinylation-streptavidin affinity in any number of methods known to the field (e.g., streptavidin beads).

### (3) Denaturing Double-Stranded DNA Molecules

In some embodiments, the method also includes (c) subjecting the amplified products to denaturing conditions. In some embodiments, denaturing conditions comprise NaOH denaturation. In some embodiments, denaturing conditions can include, but are not limited to, heat denaturation, chemical denaturation, or combinations thereof. In some embodiments, a double-stranded DNA molecule can be denatured by using heat. In some embodiments, denaturing of the double-stranded DNA molecule can be achieved by chemical denaturation. In some embodiments, chemical denaturation can include NaOH treatment. In some embodiments, the double-stranded DNA molecule can be denatured by using salt. In some embodiments, the double-stranded DNA molecule can be denatured by using salt and additional chemicals (e.g., isopropanol and ethanol).

### (4) Recovering and Generating Analyte DNA Fragments

In some embodiments, any of the methods described herein can include (d) separately recovering the adapted Watson and Crick strands and the tagged Watson and Crick strands; (e) generating a first population of analyte DNA fragments from the tagged Watson and Crick strands and generating a first sequencing read for at least one member of the first population of analyte DNA fragments; and (f) generating a second population of analyte DNA fragments from the adapted Watson and Crick strands and generating a second sequencing read for at least one member of the second population of analyte DNA fragments. In some embodiments, the recovering step (d) comprises contacting the tagged Watson and Crick strands with streptavidin-functionalized beads, and wherein the tagged Watson and Crick strands bind the streptavidin-functionalized beads.

In some embodiments, the recovered adapted Watson and Crick strands that are not bound to the streptavidin-functionalized beads are treated with bisulfite to convert Cytosine bases to Uracil bases to generate the second population of analyte DNA fragments comprising a population of converted DNA molecules. In some embodiments, the bisulfite treatment can efficiently convert C bases to U bases in DNA molecules. In some embodiments, this conversion makes the two strands (e.g., Watson and Crick strands) distinguishable. In some embodiments, the bisulfite conversion can be used to distinguish methylated C bases, which do not get converted to T bases, from unmethylated C bases, thereby illuminating epigenetic changes.

In some embodiments, the tagged Watson and Crick strands can be separated by using any pair of affinity group and its specific or particular molecule or moiety to which it binds or associates with. For example, an affinity group can be biotin which has a high affinity or preference to associate or bind to the protein avidin or streptavidin. Alternatively, an affinity group can also refer to avidin or streptavidin which has an affinity to biotin. In some embodiments, the tagged Watson and Crick strands can be selected using biotinylation-streptavidin affinity in any number of methods known to the field (e.g., streptavidin beads). Other examples of an affinity group and specific or particular molecule or moiety to which it binds or associates with include, but are not limited to, antibodies or antibody fragments and their respective antigens, such as digoxigenin and anti-digoxigenin antibodies, lectin, and carbohydrates (e.g., a sugar, a monosaccharide, a disaccharide, or a polysaccharide), and receptors and receptor ligands.

In some embodiments, the recovering step can include using magnetic beads to separate the tagged Watson and Crick strands. In some embodiments, the magnetic beads can be covalently coated with streptavidin and bound to biotinylated tagged Watson and Crick strands. In some embodiments, the magnetic beads can be purified by using a magnet. In some embodiments, the magnetic beads can be recovered by centrifugation and size fractionated through filtration or flow sorting.

In some embodiments, the tagged Watson and Crick strands can bind to single beads, wherein the beads are stained with fluorescent probes and counted using flow cytometry. Beads representing specific variants can be optionally recovered through flow sorting and used for subsequent confirmation and experimentation. In some embodiments, beads can be microspheres or microparticles. Particle sizes can vary between about 0.1 and 10 microns in diameter. Typically beads are made of a polymeric material, such as polystyrene, although nonpolymeric materials such as silica can also be used. Other materials which can be used include styrene copolymers, methyl methacrylate, functionalized polystyrene, glass, silicon, and carboxylate. Optionally the particles are superparamagnetic, which facilitates their purification after being used in reactions. In some embodiments, beads can be modified by covalent or non-covalent interactions with other materials, either to alter gross surface properties, such as hydrophobicity or hydrophilicity, or to attach molecules that impart binding specificity. Such molecules can include, but are not limited to, antibodies, ligands, members of a specific-binding protein pair, receptors, nucleic acids. Specific-binding protein pairs include avidin-biotin, streptavidin-biotin, and Factor VII-Tissue Factor.

In some embodiments, the tagged Watson and Crick strands can be separated by using treatment with a USER (Uracil-Specific Excision Reagent) enzyme, wherein the USER enzyme comprises a mixture of Uracil DNA glycosylase and the DNA glycosylase-lyase Endonuclease VIII targeting the deoxyuridine base embedded within the 5′ ends of the strands.

### Genetic Characteristic—Sequence Determination

As used herein, the term “genetic characteristic” refers to genetic information and/or material that is replicated and passed from parent to progeny cell at each cell division. In some embodiments, a genetic characteristic can be a mutation in a nucleic acid (e.g., DNA molecule). In some embodiments, the mutation is selected from the group consisting of an insertion, a deletion, a substitution, a deletion-insertion, a duplication, an inversion, a frameshift, a repeat expansion, a translocation, and combinations thereof. In some embodiments, identifying the genetic characteristic can include mutational analysis, aneuploidy analysis, or fragmentomics. Exemplary methods for identifying genetic characteristics suitable for use in the methods disclosed herein are described in PCT/US2021/017937, which is hereby incorporated by reference in its entirety.

### (a) Initial Amplification of the Adapter-Attached Templates

Following adapter attachment, the adapted double-stranded DNA molecules can be amplified (e.g., PCR amplified) in an initial amplification reaction. Any appropriate method can be used to amplify the adapted double-stranded DNA molecules. An exemplary method that can be used to amplify the adapted double-stranded DNA molecules includes, without limitation, whole-genome PCR. In some embodiments, the adapted double-stranded DNA molecule is amplified by performing a single round of linear extension. In some embodiments, the adapted double-stranded DNA molecule is amplified by performing one, two, or three round(s) of linear extension. In some embodiments, the adapted double-stranded DNA molecule is amplified by performing one or more (e.g., one, two, three, four, or five) rounds of linear extension.

Any appropriate primer pair can be used to amplify the adapted double-stranded DNA molecules. In some embodiments, a universal primer pair can be used. A primer can include, without limitation from about 12 nucleotides to about 30 nucleotides. In some embodiments, any appropriate PCR conditions can be used in the initial amplification. PCR amplification can include a denaturing phase, an annealing phase, and an extension phase. Each phase of an amplification cycle can include any appropriate conditions. In some cases, a denaturing phase can include a temperature of about 90° C. to about 105° C. (e.g., about 94° C. to about 98° C.), and a time of about 1 second to about 5 minutes (e.g., about 10 seconds to about 1 minute). For example, a denaturing phase can include a temperature of about 98° C. for about 10 seconds. In some cases, an annealing phase can include a temperature of about 50° C. to about 72° C., and a time of about 30 seconds to about 90 seconds. In some cases, an extension phase can include a temperature of about 55° C. to about 80° C., and a time of about 15 seconds per kb of the amplicon to be generated to about 30 seconds per kb of the amplicon to be generated. In some cases, annealing and extension phases can be performed in a single cycle. For example, an annealing and phase extension phase can include a temperature of about 65° C. for about 75 seconds.

PCR conditions used in the initial amplification can include any appropriate number of PCR amplification cycles. In some cases, PCR amplification can include from about 1 to about 50 (e.g., about 5 to about 50, about 10 to about 50, about 15 to about 50, about 20 to about 50, about 25 to about 50, about 30 to about 50, about 35 to about 50, about 40 to about 50, about 45 to about 50, about 1 to about 45, about 5 to about 45, about 10 to about 45, about 15 to about 45, about 20 to about 45, about 25 to about 45, about 30 to about 45, about 35 to about 45, about 40 to about 45, about 1 to about 40, about 5 to about 40, about 10 to about 40, about 15 to about 40, about 20 to about 40, about 25 to about 40, about 30 to about 40, about 35 to about 40, about 1 to about 35, about 5 to about 35, about 10 to about 35, about 15 to about 35, about 20 to about 35, about 25 to about 35, about 30 to about 35, about 1 to about 30, about 5 to about 30, about 10 to about 30, about 15 to about 30, about 20 to about 30, about 25 to about 30, about 1 to about 25, about 5 to about 25, about 10 to about 25, about 15 to about 25, about 20 to about 25, about 1 to about 20, about 5 to about 20, about 10 to about 20, about 15 to about 20, about 1 to about 15, about 5 to about 15, about 10 to about 15, about 1 to about 10, about 5 to about 10, or about 1 to about 5) cycles. In some embodiments, the PCR amplification comprises no more than 11 cycles. In some embodiments, the PCR amplification comprises no more than 7 cycles. In some embodiments, the PCR amplification comprises no more than 5 cycles.

In some cases, when PCR conditions include a heat-activated polymerase, PCR amplification also can include an initialization step. For example, PCR amplification can include an initialization step prior to performing the PCR amplification cycles. In some cases, an initialization step can include a temperature of about 94° C. to about 98° C., and a time of about 15 seconds to about 1 minute. For example, an initialization step can include a temperature of about 98° C. for about 30 seconds.

In some cases, PCR amplification also can include a hold step. For example, PCR amplification can include a hold step after performing the PCR amplification cycles, an optionally after performing any final extension step. In some case, a hold step can include a temperature of about 4° C. to about 15° C., for an indefinite amount of time.

In some cases, a duplex sequencing library generated as described herein (e.g., an amplified duplex sequencing library) can be purified. Any appropriate method can be used to purify a duplex sequencing library. An exemplary method that can be used to purify a duplex sequencing library includes, without limitation, magnetic beads (e.g., solid phase reversible immobilization (SPRI) magnetic beads).

(b) Optional ssDNA Library Prep

In some cases, a duplex sequencing library can be used to generate a library of single stranded Watson strand-derived sequences and a library of single stranded Crick-strand derived sequences. Generating a library of single stranded Watson strand-derived sequences and a library of single stranded Crick-strand derived sequences can minimize non-specific amplification (e.g., from a primer complementary to a ligated sequence such as a 3′ duplex adapter or a 5′ adapter). Any appropriate method can be used to generate a library of single stranded Watson strand-derived sequences and a library of single stranded Crick-strand derived sequences (e.g., from a duplex sequencing library generated as described herein). In some cases, a library of single stranded Watson strand-derived sequences and a library of single stranded Crick-strand derived sequences can be generated from an amplified duplex sequencing library by dividing the amplification products into at least two aliquots, and subjecting each aliquot to a PCR amplification where the Watson strand is amplified from a first aliquot, and the Crick strand is amplified from a second aliquot. For example, a first aliquot of amplification products from an amplified duplex sequencing library can be subjected to a PCR amplification using a primer pair where a first primer is biotinylated and a second primer is non-biotinylated to generate a single stranded library of Watson strands, and a second aliquot of amplification products from an amplified duplex sequencing library can be subjected to a PCR amplification using a primer pair where a first primer is non-biotinylated and a second primer is biotinylated to generate a single stranded library of Crick strands. In some cases, a library of single stranded Watson strand-derived sequences and a library of single stranded Crick-strand derived sequences can be generated.

Any appropriate method can be used to generate a library of single stranded Watson strand-derived sequences and a library of single stranded Crick-strand derived sequences from an amplified duplex sequencing library. For example, amplification products from an amplified duplex sequencing library can be separated into a first PCR amplification and a second PCR amplification in which only one of the two primers in the PCR primer pair is tagged. For example, a first PCR amplification can use a primer pair that includes a primer (e.g., a first primer) that is tagged and a primer (e.g., a second primer) that is not tagged, and a second PCR amplification can use a primer pair that includes a primer (e.g., a first primer) that is not tagged and a primer (e.g., a second primer) that is tagged. A primer tag can be any tag that enables a PCR amplification product generated from the tagged primer to be recovered. In some cases, a tagged primer can be a biotinylated primer, and a PCR amplification produce generated from the biotinylated primer can be recovered using streptavidin. In some cases, a tagged primer can be a uracil-containing biotinylated primer, and a PCR amplification produce generated from the uracil-containing biotinylated primer can be recovered using streptavidin. For example, a library of single stranded Watson strand-derived sequences and a library of single stranded Crick-strand derived sequences can be generated in a PCR amplification using a primer pair including a biotinylated primer and a non-biotinylated primer. In some cases, a tagged primer can be a phosphorylated primer, and a PCR amplification produce generated from the phosphorylated primer can be recovered using a lambda nuclease. For example, a library of single stranded Watson strand-derived sequences and a library of single stranded Crick-strand derived sequences can be generated in a PCR amplification using a primer pair including a phosphorylated primer and a non-phosphorylated primer.

Any appropriate primer pair can be used to generate a library of single stranded Watson strand-derived sequences and a library of single stranded Crick-strand derived sequences (e.g., from a duplex sequencing library generated as described herein). A primer can include, without limitation, from about 12 nucleotides to about 30 nucleotides. In some cases, a primer pair can include at least one primer that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification). Examples of primer pairs that can be used to generate a library of single stranded Watson strand-derived sequences and a library of single stranded Crick-strand derived sequences as described herein include, without limitation, a P5 primer and a P7 primer.

Any appropriate PCR conditions can be used to generate a library of single stranded Watson strand-derived sequences and a library of single stranded Crick-strand derived sequences (e.g., from a duplex sequencing library generated as described herein). PCR amplification can include a denaturing phase, an annealing phase, and an extension phase. Each phase of an amplification cycle can include any appropriate conditions. In some cases, a denaturing phase can include a temperature of about 90° C. to about 105° C., and a time of about 1 second to about 5 minutes. For example, a denaturing phase can include a temperature of about 98° C. for about 10 seconds. In some cases, an annealing phase can include a temperature of about 50° C. to about 72° C., and a time of about 30 seconds to about 90 seconds. In some cases, an extension phase can include a temperature of about 55° C. to about 80° C., and a time of about 15 seconds per kb of the amplicon to be generated to about 30 seconds per kb of the amplicon to be generated. In some cases, an extension phase reflects the processivity of the polymerase that is used. In some cases, annealing and extension phases can be performed in a single cycle. For example, an annealing and phase extension phase can include a temperature of about 65° C. for about 75 seconds.

PCR conditions used to generate a library of single stranded Watson strand-derived sequences and a library of single stranded Crick-strand derived sequences (e.g., from a duplex sequencing library generated as described herein) can include any appropriate number of PCR amplification cycles. In some cases, PCR amplification can include, without limitation, from about 1 to about 50 (e.g., about 5 to about 50, about 10 to about 50, about 15 to about 50, about 20 to about 50, about 25 to about 50, about 30 to about 50, about 35 to about 50, about 40 to about 50, about 45 to about 50, about 1 to about 45, about 5 to about 45, about 10 to about 45, about 15 to about 45, about 20 to about 45, about 25 to about 45, about 30 to about 45, about 35 to about 45, about 40 to about 45, about 1 to about 40, about 5 to about 40, about 10 to about 40, about 15 to about 40, about 20 to about 40, about 25 to about 40, about 30 to about 40, about 35 to about 40, about 1 to about 35, about 5 to about 35, about 10 to about 35, about 15 to about 35, about 20 to about 35, about 25 to about 35, about 30 to about 35, about 1 to about 30, about 5 to about 30, about 10 to about 30, about 15 to about 30, about 20 to about 30, about 25 to about 30, about 1 to about 25, about 5 to about 25, about 10 to about 25, about 15 to about 25, about 20 to about 25, about 1 to about 20, about 5 to about 20, about 10 to about 20, about 15 to about 20, about 1 to about 15, about 5 to about 15, about 10 to about 15, about 1 to about 10, about 5 to about 10, or about 1 to about 5) cycles. For example, PCR amplification can include about 4 amplification cycles. In some embodiments, PCR amplification can include about 8 amplification cycles. In some embodiments, PCT amplification can include about 11 amplification cycles.

In some cases, when PCR conditions include a heat-activated polymerase, PCR amplification also can include an initialization step. For example, PCR amplification can include an initialization step prior to performing the PCR amplification cycles. In some cases, an initialization step can include a temperature of about 94° C. to about 98° C., and a time of about 15 seconds to about 1 minute. For example, an initialization step can include a temperature of about 98° C. for about 30 seconds.

In some cases, PCR amplification also can include a hold step. For example, PCR amplification can include a hold step after performing the PCR amplification cycles, an optionally after performing any final extension step. In some case, a hold step can include a temperature of about 4° C. to about 15° C., for an indefinite amount of time.

Any appropriate method can be used to separate double stranded amplification products into single stranded amplification products. In some cases, a double stranded amplification products can be denatured to separate double stranded amplification products into two single stranded amplification products. Examples of methods that can be used to separate a double stranded amplification product into single stranded amplification products include, without limitation, heat denaturation, chemical (e.g., NaOH) denaturation, and salt denaturation.

Following PCR amplification, the tagged Watson and Crick strands can be recovered. Any appropriate method can be used to recover tagged Watson and Crick strands generated using a tagged primer. In cases where a tagged primer is a biotinylated primer, the biotinylated amplification products (e.g., generated from the biotinylated primer) can be recovered using streptavidin (e.g., streptavidin-functionalized beads). For example, when an amplified duplex sequencing library is further amplified in a first PCR amplification using a primer pair that includes a first biotinylated primer and a second non-biotinylated primer, and a second PCR amplification using a primer pair that includes a first non-biotinylated primer and a second biotinylated primer, the biotinylated amplification products generated from the first PCR amplification can be bound to streptavidin-functionalized beads (e.g., a first set of streptavidin-functionalized beads) and the biotinylated amplification products generated from the second PCR amplification can be bound to streptavidin-functionalized beads (e.g., a first second of streptavidin-functionalized beads), and the double stranded amplification products can be separated (e.g., denatured) into single strands of the amplification products. In some cases, recovering biotinylated PCR amplification products also can include releasing the biotinylated PCR amplification products from the streptavidin (e.g., the streptavidin-functionalized beads). Separating the double stranded amplification products generated by a first PCR amplification using a primer pair that includes a first biotinylated primer and a second non-biotinylated primer, and a second PCR amplification using a primer pair that includes a first non-biotinylated primer and a second biotinylated primer, can allow single stranded amplification products generated from the biotinylated primers to remain bound to the streptavidin-functionalized beads while single stranded amplification products generated from the non-biotinylated primers can be denatured (e.g., denatured and degraded) from the streptavidin-functionalized beads, thereby generating a library of single stranded Watson strand-derived sequences and a library of single stranded Crick-strand derived sequences of the duplex sequencing library.

In cases where a tagged primer is a phosphorylated primer, the phosphorylated amplification products (e.g., generated from the phosphorylated primer) can be recovered using an exonuclease (e.g., a lambda exonuclease). For example, when an amplified duplex sequencing library is further amplified in a first PCR amplification using a primer pair that includes a first phosphorylated primer and a second non-phosphorylated primer, and a second PCR amplification using a primer pair that includes a first non-phosphorylated primer and a second phosphorylated primer, the double stranded amplification products can be separated into single strands of the amplification products. Separating the double stranded amplification products generated by a first PCR amplification using a primer pair that includes a first phosphorylated primer and a second non-phosphorylated primer, and a second PCR amplification using a primer pair that includes a first non-phosphorylated primer and a second phosphorylated primer, can allow single stranded amplification products generated from the non-phosphorylated primers to be recovered while single stranded amplification products generated from the phosphorylated primers can be degraded by a lambda exonuclease, thereby generating a library of single stranded Watson strand-derived sequences and a library of single stranded Crick-strand derived sequences of the duplex sequencing library.

### (c) Target Enrichment

In some embodiments of any one of the methods herein, the amplified products are produced by the initial amplification are enriched for one or more target polynucleotides. In some embodiments, prior to target enrichment, single-stranded DNA libraries are prepared from amplified products produced by the initial amplification. Exemplary methods for producing the single-stranded DNA libraries are described herein.

Any appropriate method can be used to amplify a target region from a library of amplification products (e.g., a duplex sequencing library, a library of single stranded Watson strand-derived sequences, or a library of single stranded Crick-strand derived sequences generated as described herein). In some cases, a target region can be amplified from library of amplification products by subjecting the library of amplification products to a PCR amplification using a primer pair where a primer (e.g., a first primer) that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a primer (e.g., a second primer) that can target (e.g., target and bind to) a target region (e.g., a region of interest).

In some cases, a target region can be amplified from a library of amplification products (e.g., a duplex sequencing library, a library of single stranded Watson strand-derived sequences, or a library of single stranded Crick-strand derived sequences generated as described herein) in a single PCR amplification. For example, a target region can be amplified from a library of amplification products in a single PCR amplification using a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a target region.

In some cases, a target region can be amplified from a library of amplification products (e.g., a duplex sequencing library, a library of single stranded Watson strand-derived sequences, or a library of single stranded Crick-strand derived sequences generated as described herein) in multiple PCR amplifications. Multiple PCR amplifications (e.g., a first PCR amplification and a subsequent, nested PCR amplification) can be used to increase the specificity of amplifying a target region. For example, a target region can be amplified from a library of amplification products in a series of PCR amplifications where a first PCR amplification uses a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a target region, and subjecting the amplification products generated in the first PCR amplification to a subsequent, nested PCR amplification that uses a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a nucleic acid sequence from the target region that is present in the amplification products generated in the first PCR amplification.

Any appropriate primer pair can be used to amplify a target region from a library of amplification products (e.g., a duplex sequencing library, a library of single stranded Watson strand-derived sequences, or a library of single stranded Crick-strand derived sequences generated as described herein). A primer can include, without limitation, from about 12 nucleotides to about 30 nucleotides. In some cases, a primer pair can include a primer (e.g., a first primer) that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a primer (e.g., a second primer) that can target (e.g., target and bind to) a target region (e.g., a region of interest). Examples of primers that can target an adapter sequence containing a molecular barcode present in an amplification product generated as described herein (e.g., by ligating a 3′ duplex adapter including a first molecular barcode and a 5′ adapter including a second molecular barcode to a nucleic acid fragment in a duplex sequencing library prior to the amplification) include, without limitation, an i5 index primer and an i7 index primer. Primers that can target a target region can include a sequence that is complementary to the target region. In cases where a target region is a nucleic acid encoding TP53, examples of primers that can target nucleic acid encoding TP53 include, without limitation, TP53_342_GSP1 and TP53_GSP2.

In some cases, one or both primers of a primer pair used to amplify a target region from a library of amplification products (e.g., a duplex sequencing library, a library of single stranded Watson strand-derived sequences, or a library of single stranded Crick-strand derived sequences generated as described herein) can include one or more molecular barcodes.

In some cases, one or both primers of a primer pair used to amplify a target region from a library of amplification products (e.g., a duplex sequencing library, a library of single stranded Watson strand-derived sequences, or a library of single stranded Crick-strand derived sequences generated as described herein) can include one or more graft sequences (e.g. graft sequences for next generation sequencing).

In some embodiments, the target enrichment comprises (a) selectively amplifying amplified products of Watson strands comprising the target polynucleotide sequence with a first set of Watson target-selective primer pairs, the first set of Watson target-selective primer pairs comprising: (i) a first Watson target-selective primer comprising a sequence complementary to the R2 sequencing primer site of the universal 3′ adapter sequence, and (ii) a second Watson target-selective primer comprising a target-selective sequence, thereby creating target Watson amplification products; and (b) selectively amplifying amplified products of Crick strands comprising the same target polynucleotide sequence with a first set of Crick target-selective primer pairs, the first set of Crick target-selective primer pairs comprising: (i) a first Crick target-selective primer comprising a sequence complementary to the R1 sequencing primer site of the universal 5′ adapter sequence, and (ii) a second Crick target-selective primer comprising the same target-selective sequence as the second Watson target-selective primer sequence, thereby creating target Crick amplification products.

In some embodiments, the method further comprises purifying the target Watson amplification products and the target Crick amplification products from non-target polynucleotides. In some embodiments, the purifying comprises attaching the target Watson amplification products and the target Crick amplification products to a solid support. In some embodiments, the first Watson target-selective primer and first Crick target-selective primer comprises a first member of an affinity binding pair, and wherein the solid support comprises a second member of the affinity binding pair. In some embodiments, the first member is biotin and the second member is streptavidin. In some embodiments, the solid support comprises a bead, well, membrane, tube, column, plate, sepharose, magnetic bead, or chip. In some embodiments, the method comprises removing polynucleotides that are not attached to the solid support.

In some embodiments, the method further comprises (a) further amplifying the target Watson amplification products with a second set of Watson target-selective primers, the second set of Watson target-selective primers comprising (i) a third Watson target-selective primer comprising a sequence complementary to the R2 sequencing primer site of the universal 3′ adapter sequence, and (ii) a fourth Watson target-selective primer comprising, in the 5′ to 3′ direction, an R1 sequencing primer site and a target-selective sequence selective for the same target polynucleotide, thereby creating target Watson library members; (b) further amplifying the target Crick amplification products with a second set of Crick target-selective primers, the second set of Crick target-selective primers comprising (i) a third Crick target-selective primer comprising a sequence complementary to the R1 sequencing primer site of the universal 3′ adapter sequence, and (ii) a fourth Crick target-selective primer comprising, in the 5′ to 3′ direction, an R2 sequencing primer site and the target-selective sequence selective for the same target polynucleotide of the fourth Watson target-selective primer, thereby creating target Crick library members.

In some embodiments, the third Watson and Crick target-selective primers further comprise a sample barcode sequence. In some embodiments, the third Watson target-selective primer further comprises a first grafting sequence that enables hybridization to a first grafting primer on a sequencer and wherein the third Crick target-selective primer further comprises a second grafting sequence that enables hybridization to a second grafting primer on the sequencer. In some embodiments, the fourth Watson target-selective primer further comprises the second grafting sequence and wherein the fourth Crick target-selective primer further comprises the first grafting sequence. In some embodiments, the first grafting sequence is a P7 sequence and wherein the second grafting sequence is a P5 sequence.

Any appropriate PCR conditions can be used to generate an amplified target region as described herein (e.g., from a library of amplification products such as a duplex sequencing library, a library of single stranded Watson strand-derived sequences, or a library of single stranded Crick-strand derived sequences generated). Exemplary PCR conditions are described herein. PCR conditions used to generate an amplified target region as described herein (e.g., from a library of amplification products such as a duplex sequencing library, a library of single stranded Watson strand-derived sequences, or a library of single stranded Crick-strand derived sequences generated) can include any appropriate number of PCR amplification cycles. In some cases, PCR amplification can include, without limitation, from about 1 to about 50 (e.g., about 5 to about 50, about 10 to about 50, about 15 to about 50, about 20 to about 50, about 25 to about 50, about 30 to about 50, about 35 to about 50, about 40 to about 50, about 45 to about 50, about 1 to about 45, about 5 to about 45, about 10 to about 45, about 15 to about 45, about 20 to about 45, about 25 to about 45, about 30 to about 45, about 35 to about 45, about 40 to about 45, about 1 to about 40, about 5 to about 40, about 10 to about 40, about 15 to about 40, about 20 to about 40, about 25 to about 40, about 30 to about 40, about 35 to about 40, about 1 to about 35, about 5 to about 35, about 10 to about 35, about 15 to about 35, about 20 to about 35, about 25 to about 35, about 30 to about 35, about 1 to about 30, about 5 to about 30, about 10 to about 30, about 15 to about 30, about 20 to about 30, about 25 to about 30, about 1 to about 25, about 5 to about 25, about 10 to about 25, about 15 to about 25, about 20 to about 25, about 1 to about 20, about 5 to about 20, about 10 to about 20, about 15 to about 20, about 1 to about 15, about 5 to about 15, about 10 to about 15, about 1 to about 10, about 5 to about 10, or about 1 to about 5) cycles. For example, when PCR amplification of an amplified target region includes a single PCR amplification, the PCR amplification can include about 18 amplification cycles. For example, when PCR amplification of an amplified target region includes a first PCR amplification and a subsequent, nested PCR amplification, the first PCR amplification can include about 18 amplification cycles, and the subsequent, nested PCR amplification can include about 10 amplification cycles.

### (d) Exemplary Targets

Any appropriate target region (e.g., a region of interest) can be amplified from a library of amplification products (e.g., a duplex sequencing library, a library of single stranded Watson strand-derived sequences, or a library of single stranded Crick-strand derived sequences generated as described herein) and assessed for the presence or absence of one or more mutations. In some cases, a target region can be a region of nucleic acid in which one or more mutations are associated with a disease or disorder. Examples of target regions that can be amplified and assessed for the presence or absence of one or more mutations include, without limitation, nucleic acid encoding tumor protein p53 (TP53), nucleic acid encoding breast cancer 1 (BRCA1), nucleic acid encoding BRCA2, nucleic acid encoding a phosphatase and tensin homolog (PTEN) polypeptide, nucleic acid encoding a AKT1 polypeptide, nucleic acid encoding a APC polypeptide, nucleic acid encoding a CDKN2A polypeptide, nucleic acid encoding a EGFR polypeptide, nucleic acid encoding a FBXW7 polypeptide, nucleic acid encoding a GNAS polypeptide, nucleic acid encoding a KRAS polypeptide, nucleic acid encoding a NRAS polypeptide, nucleic acid encoding a PIK3CA polypeptide, nucleic acid encoding a BRAF polypeptide, nucleic acid encoding a CTNNB1 polypeptide, nucleic acid encoding a FGFR2 polypeptide, nucleic acid encoding a HRAS polypeptide, and nucleic acid encoding a PPP2R1A polypeptide, In some cases, a target region that can be amplified and assessed for the presence or absence of one or more mutations can be nucleic acid encoding TP53.

Any appropriate method can be used to assess a target region (e.g., an amplified target region) for the presence or absence of one or more mutations. In some cases, one or more sequencing methods can be used to assess an amplified target region for the presence or absence of one or more mutations.

### (e) Sequence Determination

In some cases, one or more sequencing methods can be used to assess an amplified target region determine whether the mutation(s) are present on both the Watson strand and the Crick strand. In some cases, sequencing reads can be used to assess an amplified target region for the presence or absence of one or more mutations and can be used to determine whether the mutation(s) are present on both the Watson strand and the Crick strand. Examples of sequencing methods that can be used to assess an amplified target region for the presence or absence of one or more mutations as describe herein include, without limitation, single read sequencing, paired-end sequencing, NGS, and deep sequencing. In some embodiments, the single read sequencing comprises sequencing across the entire length of the templates to generate the sequence reads. In some embodiments, the sequencing comprises paired end sequencing. In some embodiments, the sequencing is performed with a massively parallel sequencer. In some embodiments, the massively parallel sequencer is configured to determine sequence reads from both ends of template polynucleotides.

### (f) Analysis of Sequence Reads

In some embodiments, the sequence reads are mapped to a reference genome.

In some embodiments, the sequence reads are assigned into barcode (e.g., UID) families. A barcode family can comprise sequence reads from amplified products originating from an original template, e.g., original double-stranded DNA fragment from a nucleic acid sample.

In some embodiments, each member of a barcode family comprises the same exogenous barcode sequence. In some embodiments, each member of a barcode family further comprises the same endogenous barcode sequence. Endogenous barcodes are described herein.

In some embodiments, each member of a barcode family further comprises the same exogenous barcode sequence and the same endogenous barcode sequence. In some embodiments, the combination of the exogenous barcode sequence and endogenous barcode sequence are unique to the barcode family. In some embodiments, the combination of the exogenous barcode sequence and endogenous barcode sequence does not exist in another barcode family represented in the nucleic acid sample.

The number of members of a barcode family can depend on the depth of sequencing. In some embodiments, a barcode family comprises at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or 1000 members. In some embodiments, a UID family comprises about 2-1000 members, about 2-500 members, about 2-100 members, about 2-50 members, or about 2-20 members.

In some embodiments, the sequence reads of an individual barcode family are assigned to a Watson subfamily and a Crick subfamily. In some embodiments, the sequence reads of an individual barcode family are assigned to the Watson and Crick subfamilies based on the orientation of the insert relative to the adapter sequences. In some embodiments, the orientation of the insert relative to the adapter sequences is resolved by how the sequence reads were aligned as “read pairs” or “mate pairs”.

In some embodiments, the assignment of the sequence reads into the Watson and Crick subfamilies are based on spatial relationship of the exogenous barcode sequence to the R1 and R2 read sequence. In some embodiments, members of the Watson subfamily are characterized by the exogenous barcode sequence being downstream of the R2 sequence and upstream of the R1 sequence. In some embodiments, members of the Crick subfamily are characterized by the exogenous barcode sequence being downstream of the R1 sequence and upstream of the R2 sequence. In some embodiments, members of the Watson subfamily are characterized by the exogenous barcode sequence being in greater proximity to the R2 sequence and lesser proximity to the R1 sequence. In some embodiments, members of the Crick subfamily are characterized by the exogenous barcode sequence being in greater proximity to the R1 sequence and in lesser proximity to the R2 sequence. In some embodiments, members of the Watson subfamily are characterized by the exogenous barcode sequence being immediately downstream or within 1-70, 1-60, 1-50, 1-40, 1-30, 1-20, 1-10, or 1-5 nucleotides of the R2 sequence. In some embodiments, members of the Crick subfamily are characterized by the exogenous barcode sequence being immediately downstream or within 1-70, 1-60, 1-50, 1-40, 1-30, 1-20, 1-10, or 1-5 nucleotides of the R1 sequence.

In some embodiments, a barcode subfamily (e.g., Watson subfamily and/or Crick subfamily) comprises at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 members. In some embodiments, a barcode subfamily (e.g., Watson subfamily and/or Crick subfamily) comprises about 2-500 members, about 2-100 members, about 2-50 members, about 2-20 members, or about 2-10 members.

In some embodiments, a nucleotide sequence is determined to accurately represent a Watson strand of an analyte DNA fragment, e.g., a double stranded DNA fragment from the nucleic acid sample, when a threshold percentage (or a percentage exceeding a threshold) of members of the Watson subfamily contain the sequence. In some embodiments, a nucleotide sequence is determined to accurately represent a Crick strand of an analyte DNA fragment, e.g., a double stranded DNA fragment from the nucleic acid sample, when a threshold percentage (or a percentage exceeding a threshold) of members of the Crick subfamily contain the sequence.

Thresholds can be determined by a skilled artisan based on, e.g., number of the members of the subfamily, the particular purpose of the sequencing experiment, and the particular parameters of the sequencing experiment. In some embodiments, the threshold is set at 1%, 5%, 10%, 20%, 30%, 40%, 50%, 60%, 70%, 75%, 80%, 85%, 90%, 95%, 96%, 97%, 98%, 99%, or 100%. In particular embodiments, the threshold is set at 50%. By way of example only, in an embodiment wherein the threshold is set at 50%, a nucleotide sequence is determined to accurately represent a Watson or Crick strand of an analyte DNA fragment, e.g., a double stranded DNA fragment from the nucleic acid sample, when at least 50% of the subfamily members contain the sequence. By way of other example only, in an embodiment wherein the threshold is set at 50%, a nucleotide sequence is determined to accurately represent a Watson or Crick strand of an analyte DNA fragment, e.g., a double stranded DNA fragment from the nucleic acid sample, when more than 50% of the subfamily members contain the sequence.

In some embodiments, the sequence accurately representing the Watson strand of the analyte DNA fragment is determined to have a mutation. In some embodiments, the sequence accurately representing the Watson strand of the analyte DNA fragment is determined to have a mutation when the sequence differs from a reference sequence that lacks the mutation.

In some embodiments, the sequence accurately representing the Crick strand of the analyte DNA fragment is determined to have a mutation. In some embodiments, the sequence accurately representing the Crick strand of the analyte DNA fragment is determined to have a mutation when the sequence differs from a reference sequence that lacks the mutation.

In some embodiments, the analyte DNA fragment is determined to have the mutation when sequence accurately representing the Watson strand the sequence accurately representing the Crick strand comprise the same mutation.

In some cases, the location of the molecular barcode within the paired-end sequencing reads of the amplified target region can be used to distinguish which strand of the double stranded nucleic acid template the amplified target region was derived from. For example, when a first a paired-end sequencing read of an amplified target region indicates that a molecular barcode is read last, the amplified target region can be identified as being derived from the sense strand of the nucleic acid template, and when a first a paired-end sequencing read of an amplified target region indicates that a molecular barcode is read first, the amplified target region can be identified as being derived from the anti-sense strand of the nucleic acid template. For example, when a second a paired-end sequencing read of an amplified target region indicates that a molecular barcode is read first, the amplified target region can be identified as being derived from the anti-sense strand of the nucleic acid template, and when a second a paired-end sequencing read of an amplified target region indicates that a molecular barcode is read last, the amplified target region can be identified as being derived from the sense strand of the nucleic acid template. In some cases, paired-end sequencing can be used to distinguish amplification products derived from the Watson strand from amplification products derived from the Crick strand.

Following sequencing of target regions (e.g., target regions amplified as described herein), sequencing reads can be aligned to a reference genome and grouped by the molecular barcode present in each sequencing read. In some cases, sequencing reads that include the same molecular barcode and map to both the Watson strand and the Crick strand of the double stranded nucleic acid template (e.g., both the Watson strand and the Crick strand of the target region) can be identified as having duplex support. For example, when sequencing reads indicate the presence of one or more mutations in a target region include the same molecular barcode and map to both the Watson strand and the Crick strand of the target region, the mutation(s) can be identified as having duplex support.

Amplification of nucleic acid fragments containing a molecular barcode can be performed according to known techniques to generate families of barcoded fragments. In some embodiments, polymerase chain reaction (PCR) can be used. In some embodiments, inverse PCR may be used. In some embodiments, rolling circle amplification can be used. Amplification of fragments typically is done using primers that are complementary to priming sites that are attached to the fragments at the same time as the molecular barcodes. In some embodiments, the priming sites are distal to the molecular barcodes, so that amplification includes the molecular barcodes.

In some embodiments, amplification forms a family of fragments, each member of the family sharing the same molecular barcode. In some embodiments, the diversity of molecular barcodes present in adapter fragments is greatly in excess of the diversity of the fragments, and thus each family derives from a single nucleic acid fragment molecule. In some embodiments, primers used for the amplification may be chemically modified to render them more resistant to exonucleases. In some embodiments, family members are sequenced and compared to identify any divergences within a family. In some embodiments, sequencing is performed on a massively parallel sequencing platform, many of which are commercially available. If the sequencing platform requires a sequence for “grafting,” i.e., attachment to the sequencing device, such a sequence can be added during addition of molecular barcodes or separately. A grafting sequence may be part of a molecular barcoded primer, a universal primer, a gene target-specific primer, the amplification primers used for making a family, a sample barcoded primer, or separate. Redundant sequencing refers to the sequencing of a plurality of members of a single family.

In some embodiments, a threshold can be set for identifying a mutation in a nucleic acid fragment. If the “mutation” appears in all members of a family, then it derives from the nucleic acid fragment. If it appears in less than all members, then it may be an artifact that was introduced during the analysis (e.g., during an amplification step). Thresholds for calling a mutation may be set, for example, at 1%, 5%, 10%, 20%, 30%, 40%, 50%, 60%, 70%, 80%, 90%, 95%, 97%, 98%, or 100%. In some embodiments, the threshold for calling a mutation is 95% such that if 95% of family members sharing the same barcode include that mutation, the mutation is considered to be genuine and not an artifact. Thresholds will be set based on the number of members of a family that are sequenced and the particular purpose and situation.

In some embodiments, one or more sequencing methods can be used to assess an amplified DNA molecule and determine whether the mutation(s) are present on both strands of the double strand DNA molecule. In some embodiments, sequencing reads can be used to assess an amplified DNA molecule for the presence or absence of one or more mutations and can be used to determine whether the mutation(s) are present on both strands of the double strand DNA molecule. Examples of sequencing methods that can be used to assess an amplified DNA molecule for the presence or absence of one or more mutations as describe herein include, without limitation, single read sequencing, paired-end sequencing, NGS, and deep sequencing. In some embodiments, the single read sequencing comprises sequencing across the entire length of the templates to generate the sequence reads. In some embodiments, the sequencing comprises paired end sequencing. In some embodiments, the sequencing is performed with a massively parallel sequencer. In some embodiments, the massively parallel sequencer is configured to determine sequence reads from both ends of template polynucleotides.

In some embodiments, methods described herein include (g) grouping the first sequencing reads according to the molecular barcode present on the at least one member of the first population of analyte DNA fragments to generate a first analyte DNA family; (h) grouping the second sequencing reads according to the molecular barcode present on the at least one member of the second population of analyte DNA fragments to generate a second analyte DNA family; (i) identifying the genetic characteristic of the tagged Watson and Crick strands in the first analyte DNA family; and (j) identifying the epigenetic characteristic of the adapted Watson and Crick strands in the second analyte DNA family, thus, identifying the genetic characteristic and the epigenetic characteristic present on at least one strand of the double-stranded DNA molecule. In some embodiments, the method comprises identifying the genetic characteristic and the epigenetic characteristic present on both strands of the double-stranded DNA molecule.

### Epigenetic Characteristic—Methylation Analysis

As used herein, the term “epigenetic characteristic” can refer to a heritable phenotype change that does not involve a change in DNA sequence. In some embodiments, an epigenetic characteristic includes a functionally relevant changes to the genome that does not involve a change in the nucleotide sequence. In some embodiments, the epigenetic characteristic is hydroxymethylation, histone modification, microRNA regulation, acetylation, phosphorylation, ubiquitination, or sumoylation. In some embodiments, the epigenetic characteristic is methylation. In some embodiments, the epigenetic characteristic is a differentially methylated regions (DMR). In some embodiments, the epigenetic characteristic is a methylation pattern. In some embodiments, the methylation pattern corresponds to a methylation pattern present in cells generated via clonal hematopoiesis of indeterminate origin. In some embodiments, the methylation pattern corresponds to a methylation pattern present in a tissue of origin. In some embodiments, the tissue of origin is the anus, bladder/urothelial, breast, cervix, colon/rectum, head and neck, kidney, liver/bile duct, lung, lymphoid neoplasm, melanoma, myeloid neoplasm, ovary, pancreas/gallbladder, prostate, thyroid, upper GI, or uterus (Cypris et al., Front. Genet. 10:785 (2019), Liu et al., Ann Oncol. 31 (6): 745-759 (2020)).

In some embodiments, methods described herein can be used to detect methylation at a CpG dinucleotide in one or both strands of a double strand DNA molecule (e.g., both strands simultaneously). In some embodiments, a population of DNA molecules is treated with bisulfite to convert Cytosine bases in the DNA molecules to Uracil bases, forming a population of converted DNA molecules. In some embodiments, molecular barcodes are attached to both strands of the population of converted DNA molecules using an excess of target-specific amplification primers attached to molecular barcodes, forming a population of amplified, barcoded, converted DNA molecules. In some embodiments, the amplified, barcoded, converted DNA molecules are amplified in an amplification reaction to form families of amplified, barcoded, converted DNA molecules, wherein amplified, barcoded, converted DNA molecules that share the same molecular barcode form a family of DNA molecules. In some embodiments, a plurality of members of the families is subjected to sequencing reactions to obtain nucleotide sequences of both strands of said plurality of members of the families. In some embodiments, nucleotide sequences of a plurality of members of a family are compared and families in which >90% of the members contain a selected methylated C at a CpG dinucleotide are identified. In some embodiments, nucleotide sequences of two complementary strands of an amplified, barcoded, converted DNA molecule are compared and a methylated C at the CpG dinucleotide is identified in two complementary strands.

In some embodiments, incubation of DNA fragments with sodium bisulfite at elevated temperatures and low pH deaminates cytosine to form 5,6-dihydrocytosine-6-sulfonate. Exemplary methods of sodium bisulfite treatment for use in the methods disclosed herein are described in PCT/US2018/022664, which is hereby incorporated by reference in its entirety. Subsequent hydrolytic deamination at high pH removes the sulfonate, resulting in uracil. Many modifications of this basic reaction have been described and used largely to differentiate between cytosine and 5-methylcytosine (5-mC), the latter of which is not susceptible to bisulfite conversion. In addition to converting C to U, bisulfite treatment denatures DNA and can degrade it. Although this degradation is not limiting for standard applications of bisulfite treatment, it is critical for applications involving mutation detection in clinical samples that are already degraded prior to conversion. In some embodiments, sequencing of these products reveals that, on average, >99.8% of the C bases were converted to T bases on both strands (excluding C bases at 5′-CpG sites, which can be resistant to bisulfite conversion because they are either methylated or hydroxymethylated).

### (5) Identifying Multiple Characteristics of a Double-Stranded DNA Molecule

Also, provided herein are methods for identifying a first characteristic and a second characteristic of a double stranded DNA molecule in a population of double-stranded DNA molecules by assaying at least one strand of the double-stranded DNA molecule, the method including: (a) attaching an adapter fragment to each end of the double-stranded DNA molecule to generate an adapted double-stranded DNA molecule, wherein the adapted double-stranded DNA molecule comprises an adapted Watson strand and an adapted Crick strand, wherein the adapter fragment comprises a molecular barcode, a primer sequence, and an adapter sequence, and wherein the molecular barcode of the adapted Watson strand is the reverse complement of the molecular barcode of the adapted Crick strand; (b) copying both strands of the adapted double-stranded DNA molecule, wherein the copying comprises (i) contacting the adapted double-stranded DNA molecule with a tagged primer and (ii) performing a round of linear extension of the adapted double-stranded DNA molecule, generating a tagged Watson strand and a tagged Crick strand; (c) subjecting the amplified products to denaturing conditions; (d) separately recovering the adapted Watson and Crick strands and the tagged Watson and Crick strands; (e) generating a first population of analyte DNA fragments from the tagged Watson and Crick strands and generating a first sequencing read for at least one member of the first population of analyte DNA fragments; (f) generating a second population of analyte DNA fragments from the adapted Watson and Crick strands and generating a second sequencing read for at least one member of the second population of analyte DNA fragments; (g) grouping the first sequencing reads according to the molecular barcode present on the at least one member of the first population of analyte DNA fragments to generate a first analyte DNA family; (h) grouping the second sequencing reads according to the molecular barcode present on the at least one member of the second population of analyte DNA fragments to generate a second analyte DNA family; (i) identifying the first characteristic of the tagged Watson and Crick strands in the first analyte DNA family; and (j) identifying the second characteristic of the adapted Watson and Crick strands in the second analyte DNA family, thus, identifying the first characteristic and the second characteristic present on at least one strand of the double-stranded DNA molecule. In some embodiments, the method comprises identifying the first characteristic and the second characteristic present on both strands of the double-stranded DNA molecule.

In some embodiments, the first characteristic is a genetic characteristic. In some embodiments, the second characteristic is an epigenetic characteristic. In some embodiments, the first characteristic is a genetic characteristic or an epigenetic characteristic. In some embodiments, the second characteristic is an epigenetic characteristic or a genetic characteristic. In some embodiments, the first characteristic and second characteristic are both genetic characteristics. In some embodiments, the first characteristic and second characteristic are both epigenetic characteristic.

In some embodiments, the genetic characteristic is a mutation. In some embodiments, the mutation is selected from the group consisting of an insertion, a deletion, a substitution, a deletion-insertion, a duplication, an inversion, a frameshift, a repeat expansion, a translocation, and combinations thereof. In some embodiments, identifying the genetic characteristic comprises mutational analysis, aneuploidy analysis, or fragmentomics.

In some embodiments, the epigenetic characteristic is methylation. In some embodiments, the epigenetic characteristic is a methylation pattern. In some embodiments, the methylation pattern corresponds to a methylation pattern present in cells generated via clonal hematopoiesis of indeterminate origin. In some embodiments, the methylation pattern corresponds to a methylation pattern present in a tissue of origin. In some embodiments, the tissue of origin is the anus, bladder/urothelial, breast, cervix, colon/rectum, head and neck, kidney, liver/bile duct, lung, lymphoid neoplasm, melanoma, myeloid neoplasm, ovary, pancreas/gallbladder, prostate, thyroid, upper GI, or uterus. In some embodiments, the epigenetic characteristic is hydroxymethylation, histone modification, microRNA regulation, acetylation, phosphorylation, ubiquitination, or sumoylation.

In some embodiments, the first characteristic and second characteristic are both epigenetic characteristics, wherein the first characteristic is methylation and the second characteristic is hydroxymethylation. In some embodiments, the first characteristic is methylation and the second characteristic is acetylation. In some embodiments, the first characteristic is methylation and the second characteristic is histone modification. In some embodiments, the first characteristic is methylation and the second characteristic is microRNA regulation. In some embodiments, the first characteristic is methylation and the second characteristic is phosphorylation. In some embodiments, the first characteristic is methylation and the second characteristic is ubiquitination. In some embodiments, the first characteristic is methylation and the second characteristic is sumoylation. In some embodiments, the first characteristic is hydroxymethylation and the second characteristic is methylation. In some embodiments, the first characteristic is hydroxymethylation and the second characteristic is acetylation. In some embodiments, the first characteristic is hydroxymethylation and the second characteristic is histone modification. In some embodiments, the first characteristic is hydroxymethylation and the second characteristic is microRNA regulation. In some embodiments, the first characteristic is hydroxymethylation and the second characteristic is phosphorylation. In some embodiments, the first characteristic is hydroxymethylation and the second characteristic is ubiquitination. In some embodiments, the first characteristic is hydroxymethylation and the second characteristic is sumoylation. In some embodiments, the first characteristic is histone modification and the second characteristic is methylation. In some embodiments, the first characteristic is histone modification and the second characteristic is acetylation. In some embodiments, the first characteristic is histone modification and the second characteristic is hydroxymethylation. In some embodiments, the first characteristic is histone modification and the second characteristic is microRNA regulation. In some embodiments, the first characteristic is histone modification and the second characteristic is phosphorylation. In some embodiments, the first characteristic is histone modification and the second characteristic is ubiquitination. In some embodiments, the first characteristic is histone modification and the second characteristic is sumoylation. In some embodiments, the first characteristic is microRNA regulation and the second characteristic is methylation. In some embodiments, the first characteristic is microRNA regulation and the second characteristic is acetylation. In some embodiments, the first characteristic is microRNA regulation and the second characteristic is hydroxymethylation. In some embodiments, the first characteristic is microRNA regulation and the second characteristic is histone modification. In some embodiments, the first characteristic is microRNA regulation and the second characteristic is phosphorylation. In some embodiments, the first characteristic is microRNA regulation and the second characteristic is ubiquitination. In some embodiments, the first characteristic is microRNA regulation and the second characteristic is sumoylation. In some embodiments, the first characteristic is acetylation and the second characteristic is methylation. In some embodiments, the first characteristic is acetylation and the second characteristic is microRNA regulation. In some embodiments, the first characteristic is acetylation and the second characteristic is hydroxymethylation. In some embodiments, the first characteristic is acetylation and the second characteristic is histone modification. In some embodiments, the first characteristic is acetylation and the second characteristic is phosphorylation. In some embodiments, the first characteristic is acetylation and the second characteristic is ubiquitination. In some embodiments, the first characteristic is acetylation and the second characteristic is sumoylation, In some embodiments, the first characteristic is phosphorylation and the second characteristic is methylation. In some embodiments, the first characteristic is phosphorylation and the second characteristic is microRNA regulation. In some embodiments, the first characteristic is phosphorylation and the second characteristic is hydroxymethylation. In some embodiments, the first characteristic is phosphorylation and the second characteristic is histone modification. In some embodiments, the first characteristic is phosphorylation and the second characteristic is acetlyation. In some embodiments, the first characteristic is phosphorylation and the second characteristic is ubiquitination. In some embodiments, the first characteristic is phosphorylation and the second characteristic is sumoylation. In some embodiments, the first characteristic is ubiquitination and the second characteristic is methylation. In some embodiments, the first characteristic is ubiquitination and the second characteristic is microRNA regulation. In some embodiments, the first characteristic is ubiquitination and the second characteristic is hydroxymethylation. In some embodiments, the first characteristic is ubiquitination and the second characteristic is histone modification. In some embodiments, the first characteristic is ubiquitination and the second characteristic is acetlyation. In some embodiments, the first characteristic is ubiquitination and the second characteristic is phosphorylation. In some embodiments, the first characteristic is ubiquitination and the second characteristic is sumoylation. In some embodiments, the first characteristic is sumoylation and the second characteristic is methylation. In some embodiments, the first characteristic is sumoylation and the second characteristic is microRNA regulation. In some embodiments, the first characteristic is sumoylation and the second characteristic is hydroxymethylation. In some embodiments, the first characteristic is sumoylation and the second characteristic is histone modification. In some embodiments, the first characteristic is sumoylation and the second characteristic is acetlyation. In some embodiments, the first characteristic is sumoylation and the second characteristic is phosphorylation. In some embodiments, the first characteristic is sumoylation and the second characteristic is ubiquitination.

### Genetic Characteristic—Sequence Determination

In some embodiments, the first and/or second characteristics can be a genetic characteristic, wherein the term “genetic characteristic” refers to genetic information and/or material that is replicated and passed from parent to progeny cell at each cell division. In some embodiments, a genetic characteristic can be a mutation in a nucleic acid (e.g., DNA molecule). In some embodiments, the mutation is selected from the group consisting of an insertion, a deletion, a substitution, a deletion-insertion, a duplication, an inversion, a frameshift, a repeat expansion, a translocation, and combinations thereof. In some embodiments, identifying the genetic characteristic can include mutational analysis, aneuploidy analysis, or fragmentomics. Exemplary methods for identifying genetic characteristics suitable for use in the methods disclosed herein are described in PCT/US2021/017937, which is hereby incorporated by reference in its entirety.

### (a) Initial Amplification of the Adapter-Attached Templates

Following adapter attachment, the adapted double-stranded DNA molecules can be amplified (e.g., PCR amplified) in an initial amplification reaction. Any appropriate method can be used to amplify the adapted double-stranded DNA molecules. An exemplary method that can be used to amplify the adapted double-stranded DNA molecules includes, without limitation, whole-genome PCR.

Any appropriate primer pair can be used to amplify the adapted double-stranded DNA molecules. In some embodiments, a universal primer pair can be used. A primer can include, without limitation from about 12 nucleotides to about 30 nucleotides. In some embodiments, any appropriate PCR conditions can be used in the initial amplification. PCR amplification can include a denaturing phase, an annealing phase, and an extension phase. Each phase of an amplification cycle can include any appropriate conditions. In some cases, a denaturing phase can include a temperature of about 90° C. to about 105° C. (e.g., about 94° C. to about 98° C.), and a time of about 1 second to about 5 minutes (e.g., about 10 seconds to about 1 minute). For example, a denaturing phase can include a temperature of about 98° C. for about 10 seconds. In some cases, an annealing phase can include a temperature of about 50° C. to about 72° C., and a time of about 30 seconds to about 90 seconds. In some cases, an extension phase can include a temperature of about 55° C. to about 80° C., and a time of about 15 seconds per kb of the amplicon to be generated to about 30 seconds per kb of the amplicon to be generated. In some cases, annealing and extension phases can be performed in a single cycle. For example, an annealing and phase extension phase can include a temperature of about 65° C. for about 75 seconds.

PCR conditions used in the initial amplification can include any appropriate number of PCR amplification cycles. In some cases, PCR amplification can include from about 1 to about 50 (e.g., about 5 to about 50, about 10 to about 50, about 15 to about 50, about 20 to about 50, about 25 to about 50, about 30 to about 50, about 35 to about 50, about 40 to about 50, about 45 to about 50, about 1 to about 45, about 5 to about 45, about 10 to about 45, about 15 to about 45, about 20 to about 45, about 25 to about 45, about 30 to about 45, about 35 to about 45, about 40 to about 45, about 1 to about 40, about 5 to about 40, about 10 to about 40, about 15 to about 40, about 20 to about 40, about 25 to about 40, about 30 to about 40, about 35 to about 40, about 1 to about 35, about 5 to about 35, about 10 to about 35, about 15 to about 35, about 20 to about 35, about 25 to about 35, about 30 to about 35, about 1 to about 30, about 5 to about 30, about 10 to about 30, about 15 to about 30, about 20 to about 30, about 25 to about 30, about 1 to about 25, about 5 to about 25, about 10 to about 25, about 15 to about 25, about 20 to about 25, about 1 to about 20, about 5 to about 20, about 10 to about 20, about 15 to about 20, about 1 to about 15, about 5 to about 15, about 10 to about 15, about 1 to about 10, about 5 to about 10, or about 1 to about 5) cycles. In some embodiments, the PCR amplification comprises no more than 11 cycles. In some embodiments, the PCR amplification comprises no more than 7 cycles. In some embodiments, the PCR amplification comprises no more than 5 cycles.

In some cases, when PCR conditions include a heat-activated polymerase, PCR amplification also can include an initialization step. For example, PCR amplification can include an initialization step prior to performing the PCR amplification cycles. In some cases, an initialization step can include a temperature of about 94° C. to about 98° C., and a time of about 15 seconds to about 1 minute. For example, an initialization step can include a temperature of about 98° C. for about 30 seconds.

In some cases, PCR amplification also can include a hold step. For example, PCR amplification can include a hold step after performing the PCR amplification cycles, an optionally after performing any final extension step. In some case, a hold step can include a temperature of about 4° C. to about 15° C., for an indefinite amount of time.

In some cases, a duplex sequencing library generated as described herein (e.g., an amplified duplex sequencing library) can be purified. Any appropriate method can be used to purify a duplex sequencing library. An exemplary method that can be used to purify a duplex sequencing library includes, without limitation, magnetic beads (e.g., solid phase reversible immobilization (SPRI) magnetic beads).

(b) Optional ssDNA Library Prep

In some cases, a duplex sequencing library can be used to generate a library of single stranded Watson strand-derived sequences and a library of single stranded Crick-strand derived sequences. Generating a library of single stranded Watson strand-derived sequences and a library of single stranded Crick-strand derived sequences can minimize non-specific amplification (e.g., from a primer complementary to a ligated sequence such as a 3′ duplex adapter or a 5′ adapter). Any appropriate method can be used to generate a library of single stranded Watson strand-derived sequences and a library of single stranded Crick-strand derived sequences (e.g., from a duplex sequencing library generated as described herein). In some cases, a library of single stranded Watson strand-derived sequences and a library of single stranded Crick-strand derived sequences can be generated from an amplified duplex sequencing library by dividing the amplification products into at least two aliquots, and subjecting each aliquot to a PCR amplification where the Watson strand is amplified from a first aliquot, and the Crick strand is amplified from a second aliquot. For example, a first aliquot of amplification products from an amplified duplex sequencing library can be subjected to a PCR amplification using a primer pair where a first primer is biotinylated and a second primer is non-biotinylated to generate a single stranded library of Watson strands, and a second aliquot of amplification products from an amplified duplex sequencing library can be subjected to a PCR amplification using a primer pair where a first primer is non-biotinylated and a second primer is biotinylated to generate a single stranded library of Crick strands. In some cases, a library of single stranded Watson strand-derived sequences and a library of single stranded Crick-strand derived sequences can be generated.

Any appropriate method can be used to generate a library of single stranded Watson strand-derived sequences and a library of single stranded Crick-strand derived sequences from an amplified duplex sequencing library. For example, amplification products from an amplified duplex sequencing library can be separated into a first PCR amplification and a second PCR amplification in which only one of the two primers in the PCR primer pair is tagged. For example, a first PCR amplification can use a primer pair that includes a primer (e.g., a first primer) that is tagged and a primer (e.g., a second primer) that is not tagged, and a second PCR amplification can use a primer pair that includes a primer (e.g., a first primer) that is not tagged and a primer (e.g., a second primer) that is tagged. A primer tag can be any tag that enables a PCR amplification product generated from the tagged primer to be recovered. In some cases, a tagged primer can be a biotinylated primer, and a PCR amplification produce generated from the biotinylated primer can be recovered using streptavidin. In some cases, a tagged primer can be a uracil-containing biotinylated primer, and a PCR amplification produce generated from the uracil-containing biotinylated primer can be recovered using streptavidin. For example, a library of single stranded Watson strand-derived sequences and a library of single stranded Crick-strand derived sequences can be generated in a PCR amplification using a primer pair including a biotinylated primer and a non-biotinylated primer. In some cases, a tagged primer can be a phosphorylated primer, and a PCR amplification produce generated from the phosphorylated primer can be recovered using a lambda nuclease. For example, a library of single stranded Watson strand-derived sequences and a library of single stranded Crick-strand derived sequences can be generated in a PCR amplification using a primer pair including a phosphorylated primer and a non-phosphorylated primer.

Any appropriate primer pair can be used to generate a library of single stranded Watson strand-derived sequences and a library of single stranded Crick-strand derived sequences (e.g., from a duplex sequencing library generated as described herein). A primer can include, without limitation, from about 12 nucleotides to about 30 nucleotides. In some cases, a primer pair can include at least one primer that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification). Examples of primer pairs that can be used to generate a library of single stranded Watson strand-derived sequences and a library of single stranded Crick-strand derived sequences as described herein include, without limitation, a P5 primer and a P7 primer.

Any appropriate PCR conditions can be used to generate a library of single stranded Watson strand-derived sequences and a library of single stranded Crick-strand derived sequences (e.g., from a duplex sequencing library generated as described herein). PCR amplification can include a denaturing phase, an annealing phase, and an extension phase. Each phase of an amplification cycle can include any appropriate conditions. In some cases, a denaturing phase can include a temperature of about 90° C. to about 105° C., and a time of about 1 second to about 5 minutes. For example, a denaturing phase can include a temperature of about 98° C. for about 10 seconds. In some cases, an annealing phase can include a temperature of about 50° C. to about 72° C., and a time of about 30 seconds to about 90 seconds. In some cases, an extension phase can include a temperature of about 55° C. to about 80° C., and a time of about 15 seconds per kb of the amplicon to be generated to about 30 seconds per kb of the amplicon to be generated. In some cases, an extension phase reflects the processivity of the polymerase that is used. In some cases, annealing and extension phases can be performed in a single cycle. For example, an annealing and phase extension phase can include a temperature of about 65° C. for about 75 seconds.

PCR conditions used to generate a library of single stranded Watson strand-derived sequences and a library of single stranded Crick-strand derived sequences (e.g., from a duplex sequencing library generated as described herein) can include any appropriate number of PCR amplification cycles. In some cases, PCR amplification can include, without limitation, from about 1 to about 50 (e.g., about 5 to about 50, about 10 to about 50, about 15 to about 50, about 20 to about 50, about 25 to about 50, about 30 to about 50, about 35 to about 50, about 40 to about 50, about 45 to about 50, about 1 to about 45, about 5 to about 45, about 10 to about 45, about 15 to about 45, about 20 to about 45, about 25 to about 45, about 30 to about 45, about 35 to about 45, about 40 to about 45, about 1 to about 40, about 5 to about 40, about 10 to about 40, about 15 to about 40, about 20 to about 40, about 25 to about 40, about 30 to about 40, about 35 to about 40, about 1 to about 35, about 5 to about 35, about 10 to about 35, about 15 to about 35, about 20 to about 35, about 25 to about 35, about 30 to about 35, about 1 to about 30, about 5 to about 30, about 10 to about 30, about 15 to about 30, about 20 to about 30, about 25 to about 30, about 1 to about 25, about 5 to about 25, about 10 to about 25, about 15 to about 25, about 20 to about 25, about 1 to about 20, about 5 to about 20, about 10 to about 20, about 15 to about 20, about 1 to about 15, about 5 to about 15, about 10 to about 15, about 1 to about 10, about 5 to about 10, or about 1 to about 5) cycles. For example, PCR amplification can include about 4 amplification cycles. In some embodiments, PCR amplification can include about 8 amplification cycles.

In some cases, when PCR conditions include a heat-activated polymerase, PCR amplification also can include an initialization step. For example, PCR amplification can include an initialization step prior to performing the PCR amplification cycles. In some cases, an initialization step can include a temperature of about 94° C. to about 98° C., and a time of about 15 seconds to about 1 minute. For example, an initialization step can include a temperature of about 98° C. for about 30 seconds.

In some cases, PCR amplification also can include a hold step. For example, PCR amplification can include a hold step after performing the PCR amplification cycles, an optionally after performing any final extension step. In some case, a hold step can include a temperature of about 4° C. to about 15° C., for an indefinite amount of time.

Any appropriate method can be used to separate double stranded amplification products into single stranded amplification products. In some cases, a double stranded amplification products can be denatured to separate double stranded amplification products into two single stranded amplification products. Examples of methods that can be used to separate a double stranded amplification product into single stranded amplification products include, without limitation, heat denaturation, chemical (e.g., NaOH) denaturation, and salt denaturation.

Following PCR amplification, the tagged Watson and Crick strands can be recovered. Any appropriate method can be used to recover tagged Watson and Crick strands generated using a tagged primer. In cases where a tagged primer is a biotinylated primer, the biotinylated amplification products (e.g., generated from the biotinylated primer) can be recovered using streptavidin (e.g., streptavidin-functionalized beads). For example, when an amplified duplex sequencing library is further amplified in a first PCR amplification using a primer pair that includes a first biotinylated primer and a second non-biotinylated primer, and a second PCR amplification using a primer pair that includes a first non-biotinylated primer and a second biotinylated primer, the biotinylated amplification products generated from the first PCR amplification can be bound to streptavidin-functionalized beads (e.g., a first set of streptavidin-functionalized beads) and the biotinylated amplification products generated from the second PCR amplification can be bound to streptavidin-functionalized beads (e.g., a first second of streptavidin-functionalized beads), and the double stranded amplification products can be separated (e.g., denatured) into single strands of the amplification products. In some cases, recovering biotinylated PCR amplification products also can include releasing the biotinylated PCR amplification products from the streptavidin (e.g., the streptavidin-functionalized beads). Separating the double stranded amplification products generated by a first PCR amplification using a primer pair that includes a first biotinylated primer and a second non-biotinylated primer, and a second PCR amplification using a primer pair that includes a first non-biotinylated primer and a second biotinylated primer, can allow single stranded amplification products generated from the biotinylated primers to remain bound to the streptavidin-functionalized beads while single stranded amplification products generated from the non-biotinylated primers can be denatured (e.g., denatured and degraded) from the streptavidin-functionalized beads, thereby generating a library of single stranded Watson strand-derived sequences and a library of single stranded Crick-strand derived sequences of the duplex sequencing library.

In cases where a tagged primer is a phosphorylated primer, the phosphorylated amplification products (e.g., generated from the phosphorylated primer) can be separated from the non-phosphorylated amplification products by using an exonuclease (e.g., a lambda exonuclease). For example, when an amplified duplex sequencing library is further amplified in a first PCR amplification using a primer pair that includes a first phosphorylated primer and a second non-phosphorylated primer, and a second PCR amplification using a primer pair that includes a first non-phosphorylated primer and a second phosphorylated primer, the double stranded amplification products can be separated into single strands of the amplification products. Separating the double stranded amplification products generated by a first PCR amplification using a primer pair that includes a first phosphorylated primer and a second non-phosphorylated primer, and a second PCR amplification using a primer pair that includes a first non-phosphorylated primer and a second phosphorylated primer, can allow single stranded amplification products generated from the non-phosphorylated primers to be recovered while single stranded amplification products generated from the phosphorylated primers can be degraded by a lambda exonuclease, thereby generating a library of single stranded Watson strand-derived sequences and a library of single stranded Crick-strand derived sequences of the duplex sequencing library.

### (c) Target Enrichment

In some embodiments of any one of the methods herein, the amplified products are produced by the initial amplification are enriched for one or more target polynucleotides. In some embodiments, prior to target enrichment, single-stranded DNA libraries are prepared from amplified products produced by the initial amplification. Exemplary methods for producing the single-stranded DNA libraries are described herein.

Any appropriate method can be used to amplify a target region from a library of amplification products (e.g., a duplex sequencing library, a library of single stranded Watson strand-derived sequences, or a library of single stranded Crick-strand derived sequences generated as described herein). In some cases, a target region can be amplified from library of amplification products by subjecting the library of amplification products to a PCR amplification using a primer pair where a primer (e.g., a first primer) that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a primer (e.g., a second primer) that can target (e.g., target and bind to) a target region (e.g., a region of interest).

In some cases, a target region can be amplified from a library of amplification products (e.g., a duplex sequencing library, a library of single stranded Watson strand-derived sequences, or a library of single stranded Crick-strand derived sequences generated as described herein) in a single PCR amplification. For example, a target region can be amplified from a library of amplification products in a single PCR amplification using a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a target region.

In some cases, a target region can be amplified from a library of amplification products (e.g., a duplex sequencing library, a library of single stranded Watson strand-derived sequences, or a library of single stranded Crick-strand derived sequences generated as described herein) in multiple PCR amplifications. Multiple PCR amplifications (e.g., a first PCR amplification and a subsequent, nested PCR amplification) can be used to increase the specificity of amplifying a target region. For example, a target region can be amplified from a library of amplification products in a series of PCR amplifications where a first PCR amplification uses a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a target region, and subjecting the amplification products generated in the first PCR amplification to a subsequent, nested PCR amplification that uses a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a nucleic acid sequence from the target region that is present in the amplification products generated in the first PCR amplification.

Any appropriate primer pair can be used to amplify a target region from a library of amplification products (e.g., a duplex sequencing library, a library of single stranded Watson strand-derived sequences, or a library of single stranded Crick-strand derived sequences generated as described herein). A primer can include, without limitation, from about 12 nucleotides to about 30 nucleotides. In some cases, a primer pair can include a primer (e.g., a first primer) that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a primer (e.g., a second primer) that can target (e.g., target and bind to) a target region (e.g., a region of interest). Examples of primers that can target an adapter sequence containing a molecular barcode present in an amplification product generated as described herein (e.g., by ligating a 3′ duplex adapter including a first molecular barcode and a 5′ adapter including a second molecular barcode to a nucleic acid fragment in a duplex sequencing library prior to the amplification) include, without limitation, an i5 index primer and an i7 index primer. Primers that can target a target region can include a sequence that is complementary to the target region. In cases where a target region is a nucleic acid encoding TP53, examples of primers that can target nucleic acid encoding TP53 include, without limitation, TP53_342_GSP1 and TP53_GSP2.

In some cases, one or both primers of a primer pair used to amplify a target region from a library of amplification products (e.g., a duplex sequencing library, a library of single stranded Watson strand-derived sequences, or a library of single stranded Crick-strand derived sequences generated as described herein) can include one or more molecular barcodes.

In some cases, one or both primers of a primer pair used to amplify a target region from a library of amplification products (e.g., a duplex sequencing library, a library of single stranded Watson strand-derived sequences, or a library of single stranded Crick-strand derived sequences generated as described herein) can include one or more graft sequences (e.g. graft sequences for next generation sequencing).

In some embodiments, the target enrichment comprises (a) selectively amplifying amplified products of Watson strands comprising the target polynucleotide sequence with a first set of Watson target-selective primer pairs, the first set of Watson target-selective primer pairs comprising: (i) a first Watson target-selective primer comprising a sequence complementary to the R2 sequencing primer site of the universal 3′ adapter sequence, and (ii) a second Watson target-selective primer comprising a target-selective sequence, thereby creating target Watson amplification products; and (b) selectively amplifying amplified products of Crick strands comprising the same target polynucleotide sequence with a first set of Crick target-selective primer pairs, the first set of Crick target-selective primer pairs comprising: (i) a first Crick target-selective primer comprising a sequence complementary to the R1 sequencing primer site of the universal 5′ adapter sequence, and (ii) a second Crick target-selective primer comprising the same target-selective sequence as the second Watson target-selective primer sequence, thereby creating target Crick amplification products.

In some embodiments, the method further comprises purifying the target Watson amplification products and the target Crick amplification products from non-target polynucleotides. In some embodiments, the purifying comprises attaching the target Watson amplification products and the target Crick amplification products to a solid support. In some embodiments, the first Watson target-selective primer and first Crick target-selective primer comprises a first member of an affinity binding pair, and wherein the solid support comprises a second member of the affinity binding pair. In some embodiments, the first member is biotin and the second member is streptavidin. In some embodiments, the solid support comprises a bead, well, membrane, tube, column, plate, sepharose, magnetic bead, or chip. In some embodiments, the method comprises removing polynucleotides that are not attached to the solid support.

In some embodiments, the method further comprises (a) further amplifying the target Watson amplification products with a second set of Watson target-selective primers, the second set of Watson target-selective primers comprising (i) a third Watson target-selective primer comprising a sequence complementary to the R2 sequencing primer site of the universal 3′ adapter sequence, and (ii) a fourth Watson target-selective primer comprising, in the 5′ to 3′ direction, an R1 sequencing primer site and a target-selective sequence selective for the same target polynucleotide, thereby creating target Watson library members; (b) further amplifying the target Crick amplification products with a second set of Crick target-selective primers, the second set of Crick target-selective primers comprising (i) a third Crick target-selective primer comprising a sequence complementary to the R1 sequencing primer site of the universal 3′ adapter sequence, and (ii) a fourth Crick target-selective primer comprising, in the 5′ to 3′ direction, an R2 sequencing primer site and the target-selective sequence selective for the same target polynucleotide of the fourth Watson target-selective primer, thereby creating target Crick library members.

In some embodiments, the third Watson and Crick target-selective primers further comprise a sample barcode sequence. In some embodiments, the third Watson target-selective primer further comprises a first grafting sequence that enables hybridization to a first grafting primer on a sequencer and wherein the third Crick target-selective primer further comprises a second grafting sequence that enables hybridization to a second grafting primer on the sequencer. In some embodiments, the fourth Watson target-selective primer further comprises the second grafting sequence and wherein the fourth Crick target-selective primer further comprises the first grafting sequence. In some embodiments, the first grafting sequence is a P7 sequence and wherein the second grafting sequence is a P5 sequence.

Any appropriate PCR conditions can be used to generate an amplified target region as described herein (e.g., from a library of amplification products such as a duplex sequencing library, a library of single stranded Watson strand-derived sequences, or a library of single stranded Crick-strand derived sequences generated). Exemplary PCR conditions are described herein. PCR conditions used to generate an amplified target region as described herein (e.g., from a library of amplification products such as a duplex sequencing library, a library of single stranded Watson strand-derived sequences, or a library of single stranded Crick-strand derived sequences generated) can include any appropriate number of PCR amplification cycles. In some cases, PCR amplification can include, without limitation, from about 1 to about 50 (e.g., about 5 to about 50, about 10 to about 50, about 15 to about 50, about 20 to about 50, about 25 to about 50, about 30 to about 50, about 35 to about 50, about 40 to about 50, about 45 to about 50, about 1 to about 45, about 5 to about 45, about 10 to about 45, about 15 to about 45, about 20 to about 45, about 25 to about 45, about 30 to about 45, about 35 to about 45, about 40 to about 45, about 1 to about 40, about 5 to about 40, about 10 to about 40, about 15 to about 40, about 20 to about 40, about 25 to about 40, about 30 to about 40, about 35 to about 40, about 1 to about 35, about 5 to about 35, about 10 to about 35, about 15 to about 35, about 20 to about 35, about 25 to about 35, about 30 to about 35, about 1 to about 30, about 5 to about 30, about 10 to about 30, about 15 to about 30, about 20 to about 30, about 25 to about 30, about 1 to about 25, about 5 to about 25, about 10 to about 25, about 15 to about 25, about 20 to about 25, about 1 to about 20, about 5 to about 20, about 10 to about 20, about 15 to about 20, about 1 to about 15, about 5 to about 15, about 10 to about 15, about 1 to about 10, about 5 to about 10, or about 1 to about 5) cycles. For example, when PCR amplification of an amplified target region includes a single PCR amplification, the PCR amplification can include about 18 amplification cycles. For example, when PCR amplification of an amplified target region includes a first PCR amplification and a subsequent, nested PCR amplification, the first PCR amplification can include about 18 amplification cycles, and the subsequent, nested PCR amplification can include about 10 amplification cycles.

### (d) Exemplary Targets

Any appropriate target region (e.g., a region of interest) can be amplified from a library of amplification products (e.g., a duplex sequencing library, a library of single stranded Watson strand-derived sequences, or a library of single stranded Crick-strand derived sequences generated as described herein) and assessed for the presence or absence of one or more mutations. In some cases, a target region can be a region of nucleic acid in which one or more mutations are associated with a disease or disorder. Examples of target regions that can be amplified and assessed for the presence or absence of one or more mutations include, without limitation, nucleic acid encoding tumor protein p53 (TP53), nucleic acid encoding breast cancer 1 (BRCA1), nucleic acid encoding BRCA2, nucleic acid encoding a phosphatase and tensin homolog (PTEN) polypeptide, nucleic acid encoding a AKT1 polypeptide, nucleic acid encoding a APC polypeptide, nucleic acid encoding a CDKN2A polypeptide, nucleic acid encoding a EGFR polypeptide, nucleic acid encoding a FBXW7 polypeptide, nucleic acid encoding a GNAS polypeptide, nucleic acid encoding a KRAS polypeptide, nucleic acid encoding a NRAS polypeptide, nucleic acid encoding a PIK3CA polypeptide, nucleic acid encoding a BRAF polypeptide, nucleic acid encoding a CTNNB1 polypeptide, nucleic acid encoding a FGFR2 polypeptide, nucleic acid encoding a HRAS polypeptide, and nucleic acid encoding a PPP2R1A polypeptide, In some cases, a target region that can be amplified and assessed for the presence or absence of one or more mutations can be nucleic acid encoding TP53.

Any appropriate method can be used to assess a target region (e.g., an amplified target region) for the presence or absence of one or more mutations. In some cases, one or more sequencing methods can be used to assess an amplified target region for the presence or absence of one or more mutations.

### (e) Sequence Determination

In some cases, one or more sequencing methods can be used to assess an amplified target region determine whether the mutation(s) are present on both the Watson strand and the Crick strand. In some cases, sequencing reads can be used to assess an amplified target region for the presence or absence of one or more mutations and can be used to determine whether the mutation(s) are present on both the Watson strand and the Crick strand. Examples of sequencing methods that can be used to assess an amplified target region for the presence or absence of one or more mutations as describe herein include, without limitation, single read sequencing, paired-end sequencing, NGS, and deep sequencing. In some embodiments, the single read sequencing comprises sequencing across the entire length of the templates to generate the sequence reads. In some embodiments, the sequencing comprises paired end sequencing. In some embodiments, the sequencing is performed with a massively parallel sequencer. In some embodiments, the massively parallel sequencer is configured to determine sequence reads from both ends of template polynucleotides. In some embodiments, the sequencing comprises whole-genome PCR, whole-genome bisulfite sequencing, or capture sequencing.

### (f) Analysis of Sequence Reads

In some embodiments, the sequence reads are mapped to a reference genome.

In some embodiments, the sequence reads are assigned into barcode (e.g., UID) families. A barcode family can comprise sequence reads from amplified products originating from an original template, e.g., original double-stranded DNA fragment from a nucleic acid sample. In some embodiments, each member of a barcode family comprises the same exogenous barcode sequence. In some embodiments, each member of a barcode family further comprises the same endogenous barcode sequence. Endogenous barcodes are described herein.

In some embodiments, each member of a barcode family further comprises the same exogenous barcode sequence and the same endogenous barcode sequence. In some embodiments, the combination of the exogenous barcode sequence and endogenous barcode sequence are unique to the barcode family. In some embodiments, the combination of the exogenous barcode sequence and endogenous barcode sequence does not exist in another barcode family represented in the nucleic acid sample.

The number of members of a barcode family can depend on the depth of sequencing. In some embodiments, a barcode family comprises at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or 1000 members. In some embodiments, a UID family comprises about 2-1000 members, about 2-500 members, about 2-100 members, about 2-50 members, or about 2-20 members.

In some embodiments, the sequence reads of an individual barcode family are assigned to a Watson subfamily and a Crick subfamily. In some embodiments, the sequence reads of an individual barcode family are assigned to the Watson and Crick subfamilies based on the orientation of the insert relative to the adapter sequences. In some embodiments, the orientation of the insert relative to the adapter sequences is resolved by how the sequence reads were aligned as “read pairs” or “mate pairs”.

In some embodiments, the assignment of the sequence reads into the Watson and Crick subfamilies are based on spatial relationship of the exogenous barcode sequence to the R1 and R2 read sequence. In some embodiments, members of the Watson subfamily are characterized by the exogenous barcode sequence being downstream of the R2 sequence and upstream of the R1 sequence. In some embodiments, members of the Crick subfamily are characterized by the exogenous barcode sequence being downstream of the R1 sequence and upstream of the R2 sequence. In some embodiments, members of the Watson subfamily are characterized by the exogenous barcode sequence being in greater proximity to the R2 sequence and lesser proximity to the R1 sequence. In some embodiments, members of the Crick subfamily are characterized by the exogenous barcode sequence being in greater proximity to the R1 sequence and in lesser proximity to the R2 sequence. In some embodiments, members of the Watson subfamily are characterized by the exogenous barcode sequence being immediately downstream or within 1-70, 1-60, 1-50, 1-40, 1-30, 1-20, 1-10, or 1-5 nucleotides of the R2 sequence. In some embodiments, members of the Crick subfamily are characterized by the exogenous barcode sequence being immediately downstream or within 1-70, 1-60, 1-50, 1-40, 1-30, 1-20, 1-10, or 1-5 nucleotides of the R1 sequence.

In some embodiments, a barcode subfamily (e.g., Watson subfamily and/or Crick subfamily) comprises at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 members. In some embodiments, a barcode subfamily (e.g., Watson subfamily and/or Crick subfamily) comprises about 2-500 members, about 2-100 members, about 2-50 members, about 2-20 members, or about 2-10 members.

In some embodiments, a nucleotide sequence is determined to accurately represent a Watson strand of an analyte DNA fragment, e.g., a double stranded DNA fragment from the nucleic acid sample, when a threshold percentage (or a percentage exceeding a threshold) of members of the Watson subfamily contain the sequence. In some embodiments, a nucleotide sequence is determined to accurately represent a Crick strand of an analyte DNA fragment, e.g., a double stranded DNA fragment from the nucleic acid sample, when a threshold percentage (or a percentage exceeding a threshold) of members of the Crick subfamily contain the sequence. Thresholds can be determined by a skilled artisan based on, e.g., number of the members of the subfamily, the particular purpose of the sequencing experiment, and the particular parameters of the sequencing experiment. In some embodiments, the threshold is set at 1%, 5%, 10%, 20%, 30%, 40%, 50%, 60%, 70%, 75%, 80%, 85%, 90%, 95%, 96%, 97%, 98%, 99%, or 100%. In particular embodiments, the threshold is set at 50%. By way of example only, in an embodiment wherein the threshold is set at 50%, a nucleotide sequence is determined to accurately represent a Watson or Crick strand of an analyte DNA fragment, e.g., a double stranded DNA fragment from the nucleic acid sample, when at least 50% of the subfamily members contain the sequence. By way of other example only, in an embodiment wherein the threshold is set at 50%, a nucleotide sequence is determined to accurately represent a Watson or Crick strand of an analyte DNA fragment, e.g., a double stranded DNA fragment from the nucleic acid sample, when more than 50% of the subfamily members contain the sequence.

In some embodiments, the sequence accurately representing the Watson strand of the analyte DNA fragment is determined to have a mutation. In some embodiments, the sequence accurately representing the Watson strand of the analyte DNA fragment is determined to have a mutation when the sequence differs from a reference sequence that lacks the mutation.

In some embodiments, the sequence accurately representing the Crick strand of the analyte DNA fragment is determined to have a mutation. In some embodiments, the sequence accurately representing the Crick strand of the analyte DNA fragment is determined to have a mutation when the sequence differs from a reference sequence that lacks the mutation.

In some embodiments, the analyte DNA fragment is determined to have the mutation when sequence accurately representing the Watson strand the sequence accurately representing the Crick strand comprise the same mutation.

In some cases, the location of the molecular barcode within the paired-end sequencing reads of the amplified target region can be used to distinguish which strand of the double stranded nucleic acid template the amplified target region was derived from. For example, when a first a paired-end sequencing read of an amplified target region indicates that a molecular barcode is read last, the amplified target region can be identified as being derived from the sense strand of the nucleic acid template, and when a first a paired-end sequencing read of an amplified target region indicates that a molecular barcode is read first, the amplified target region can be identified as being derived from the anti-sense strand of the nucleic acid template. For example, when a second a paired-end sequencing read of an amplified target region indicates that a molecular barcode is read first, the amplified target region can be identified as being derived from the anti-sense strand of the nucleic acid template, and when a second a paired-end sequencing read of an amplified target region indicates that a molecular barcode is read last, the amplified target region can be identified as being derived from the sense strand of the nucleic acid template. In some cases, paired-end sequencing can be used to distinguish amplification products derived from the Watson strand from amplification products derived from the Crick strand.

Following sequencing of target regions (e.g., target regions amplified as described herein), sequencing reads can be aligned to a reference genome and grouped by the molecular barcode present in each sequencing read. In some cases, sequencing reads that include the same molecular barcode and map to both the Watson strand and the Crick strand of the double stranded nucleic acid template (e.g., both the Watson strand and the Crick strand of the target region) can be identified as having duplex support. For example, when sequencing reads indicate the presence of one or more mutations in a target region include the same molecular barcode and map to both the Watson strand and the Crick strand of the target region, the mutation(s) can be identified as having duplex support.

Amplification of nucleic acid fragments containing a molecular barcode can be performed according to known techniques to generate families of barcoded fragments. In some embodiments, polymerase chain reaction (PCR) can be used. In some embodiments, inverse PCR may be used. In some embodiments, rolling circle amplification can be used. Amplification of fragments typically is done using primers that are complementary to priming sites that are attached to the fragments at the same time as the molecular barcodes. In some embodiments, the priming sites are distal to the molecular barcodes, so that amplification includes the molecular barcodes.

In some embodiments, amplification forms a family of fragments, each member of the family sharing the same molecular barcode. In some embodiments, the diversity of molecular barcodes present in adapter fragments is greatly in excess of the diversity of the fragments, and thus each family derives from a single nucleic acid fragment molecule. In some embodiments, primers used for the amplification may be chemically modified to render them more resistant to exonucleases. In some embodiments, family members are sequenced and compared to identify any divergences within a family. In some embodiments, sequencing is performed on a massively parallel sequencing platform, many of which are commercially available. If the sequencing platform requires a sequence for “grafting,” i.e., attachment to the sequencing device, such a sequence can be added during addition of molecular barcodes or separately. A grafting sequence may be part of a molecular barcoded primer, a universal primer, a gene target-specific primer, the amplification primers used for making a family, a sample barcoded primer, or separate. Redundant sequencing refers to the sequencing of a plurality of members of a single family.

In some embodiments, a threshold can be set for identifying a mutation in a nucleic acid fragment. If the “mutation” appears in all members of a family, then it derives from the nucleic acid fragment. If it appears in less than all members, then it may be an artifact that was introduced during the analysis (e.g., during an amplification step). Thresholds for calling a mutation may be set, for example, at 1%, 5%, 10%, 20%, 30%, 40%, 50%, 60%, 70%, 80%, 90%, 95%, 97%, 98%, or 100%. In some embodiments, the threshold for calling a mutation is 95% such that if 95% of family members sharing the same barcode include that mutation, the mutation is considered to be genuine and not an artifact. Thresholds will be set based on the number of members of a family that are sequenced and the particular purpose and situation.

In some embodiments, one or more sequencing methods can be used to assess an amplified DNA molecule and determine whether the mutation(s) are present on both strands of the double strand DNA molecule. In some embodiments, sequencing reads can be used to assess an amplified DNA molecule for the presence or absence of one or more mutations and can be used to determine whether the mutation(s) are present on both strands of the double strand DNA molecule. Examples of sequencing methods that can be used to assess an amplified DNA molecule for the presence or absence of one or more mutations as describe herein include, without limitation, single read sequencing, paired-end sequencing, NGS, and deep sequencing. In some embodiments, the single read sequencing comprises sequencing across the entire length of the templates to generate the sequence reads. In some embodiments, the sequencing comprises paired end sequencing. In some embodiments, the sequencing is performed with a massively parallel sequencer. In some embodiments, the massively parallel sequencer is configured to determine sequence reads from both ends of template polynucleotides.

In some embodiments, methods described herein include (g) grouping the first sequencing reads according to the molecular barcode present on the at least one member of the first population of analyte DNA fragments to generate a first analyte DNA family; (h) grouping the second sequencing reads according to the molecular barcode present on the at least one member of the second population of analyte DNA fragments to generate a second analyte DNA family; (i) identifying the first characteristic of the tagged Watson and Crick strands in the first analyte DNA family; and (j) identifying the second characteristic of the adapted Watson and Crick strands in the second analyte DNA family, thus, identifying the first characteristic and the second characteristic present on at least one strand of the double-stranded DNA molecule. In some embodiments, the method comprises identifying the first characteristic and the second characteristic present on both strands of the double-stranded DNA molecule.

### Epigenetic Characteristic—Methylation Analysis

In some embodiments, the first and/or second characteristics can be an epigenetic characteristic, wherein the term “epigenetic characteristic” can refer to a heritable phenotype change that does not involve a change in DNA sequence. In some embodiments, an epigenetic characteristic includes a functionally relevant changes to the genome that does not involve a change in the nucleotide sequence. In some embodiments, the epigenetic characteristic is hydroxymethylation, histone modification, microRNA regulation, acetylation, phosphorylation, ubiquitination, or sumoylation. In some embodiments, the epigenetic characteristic is methylation. In some embodiments, the epigenetic characteristic is a methylation pattern. In some embodiments, the methylation pattern corresponds to a methylation pattern present in cells generated via clonal hematopoiesis of indeterminate origin. In some embodiments, the methylation pattern corresponds to a methylation pattern present in a tissue of origin. In some embodiments, the tissue of origin is the anus, bladder/urothelial, breast, cervix, colon/rectum, head and neck, kidney, liver/bile duct, lung, lymphoid neoplasm, melanoma, myeloid neoplasm, ovary, pancreas/gallbladder, prostate, thyroid, upper GI, or uterus (Cypris et al., Front. Genet. 10:785 (2019), Liu et al., Ann Oncol. 31 (6): 745-759 (2020)).

In some embodiments, methods described herein can be used to detect methylation at a CpG dinucleotide in one or both strands of a double strand DNA molecule (e.g., both strands simultaneously). In some embodiments, a population of DNA molecules is treated with bisulfite to convert Cytosine bases in the DNA molecules to Uracil bases, forming a population of converted DNA molecules. In some embodiments, molecular barcodes are attached to both strands of the population of converted DNA molecules using an excess of target-specific amplification primers attached to molecular barcodes, forming a population of amplified, barcoded, converted DNA molecules. In some embodiments, the amplified, barcoded, converted DNA molecules are amplified in an amplification reaction to form families of amplified, barcoded, converted DNA molecules, wherein amplified, barcoded, converted DNA molecules that share the same molecular barcode form a family of DNA molecules. In some embodiments, a plurality of members of the families is subjected to sequencing reactions to obtain nucleotide sequences of both strands of said plurality of members of the families. In some embodiments, nucleotide sequences of a plurality of members of a family are compared and families in which >90% of the members contain a selected methylated C at a CpG dinucleotide are identified. In some embodiments, nucleotide sequences of two complementary strands of an amplified, barcoded, converted DNA molecule are compared and a methylated C at the CpG dinucleotide is identified in two complementary strands.

In some embodiments, incubation of DNA fragments with sodium bisulfite at elevated temperatures and low pH deaminates cytosine to form 5,6-dihydrocytosine-6-sulfonate. Exemplary methods of sodium bisulfite treatment for use in the methods disclosed herein are described in PCT/US2018/022664, which is hereby incorporated by reference in its entirety. Subsequent hydrolytic deamination at high pH removes the sulfonate, resulting in uracil. Many modifications of this basic reaction have been described and used largely to differentiate between cytosine and 5-methylcytosine (5-mC), the latter of which is not susceptible to bisulfite conversion. In addition to converting C to U, bisulfite treatment denatures DNA and can degrade it. Although this degradation is not limiting for standard applications of bisulfite treatment, it is critical for applications involving mutation detection in clinical samples that are already degraded prior to conversion. In some embodiments, sequencing of these products reveals that, on average, >99.8% of the C bases were converted to T bases on both strands (excluding C bases at 5′-CpG sites, which can be resistant to bisulfite conversion because they are either methylated or hydroxymethylated).

### EXAMPLES

The disclosure is further described in the following examples, which do not limit the scope of the disclosure described in the claims.

### Example 1—Bisulfite Treatment, Library Preparation, and Sequencing

The EZ DNA Methylation Kit (Zymo Research, cat. no. D5001) was chosen to bisulfite treat and desulphonate DNA samples following the manufacturer's recommended protocol. DNA was denatured in dilute M-Dilution buffer at 37° C. for 15 minutes then bisulfite converted in the dark at 50° C. for 16 hours before being placed on ice for 10 min. After a single wash with M-Wash buffer, the sample was desulphonated for 15 min at room temperature. The sample was washed twice in M-Wash Buffer then eluted in 15 μL of Elution Buffer and stored at −20° C. Next generation sequencing libraries were prepared using the Accel-NGS Methyl-Seq DNA Library kit (Swift Bioscience, Catalog #30024), with 9 PCR cycles used at the indexing stage. Each library was paired-end sequenced to 150 bp on a single lane of an Illumina HiSeq 4000 instrument. Reads passing Illumina CASAVA Chastity filters were used for subsequent analysis. FASTQ files from the bisulfite sequencing can be obtained from the European Genome-phenome Archive.

### Example 2—DNA Sequencing Data Analysis

Illumina adapters and bases with quality scores below 25 were trimmed from the head and tail of each read using Trimmomatic. To allow for whole genome alignment to hg19, the 14 bp UID and 13 bp constant sequence were trimmed from the heads of Reads 1 and 2 using Trimmomatic v0.38. BSMAP was used to align each paired-end read to the bisulfite-converted hg19 genome, and the average methylation at each CpG computed using BSMAP's methratio.py script.

### Example 3—Identification of Methylation Markers for Plasma cfDNA Tissue Deconvolution

The average contribution of twelve tissue types (liver, lungs, colon, small intestines, pancreas, adrenal glands, esophagus, heart, brain, T cells, B cells, and neutrophils) to the total cfDNA pool was determined using 5,653 differentially methylated 500 bp regions. The bisulfite sequencing data for 12 human tissues were analyzed to identify methylation markers for plasma DNA tissue mapping. Whole genome bisulfite sequencing data for the liver, lungs, esophagus, heart, pancreas, colon, small intestines, adrenal glands, brain, and T cells were retrieved from the Human Epigenome Atlas from the Baylor College of Medicine (www.genboree.org/epigenomeatlas/index.rhtml).

All CpG islands (CGIs) and CpG shores on autosomes were assessed for potential inclusion into the methylation marker set. CGIs and CpG shores on sex chromosomes were not used, to minimize potential variations in methylation levels related to the sex-associated chromosome dosage difference in the source data. CGIs were downloaded from the University of California, Santa Cruz (UCSC) database (genome.ucsc.edu/, 27,048 CGIs for the human genome), and CpG shores were defined as 2-kb flanking windows of the CGIs. Then, the CGIs and CpG shores were subdivided into nonoverlapping 500-bp units, and each unit was considered a potential methylation marker.

The methylation densities (i.e., the percentage of CpGs being methylated within a 500-bp unit) of all of the potential marker loci were compared between the 12 tissue types. Using the methylation profiles of the 12 tissue types, two types of methylation markers were identified. Type I markers refer to any genomic loci with methylation densities that are 3 SDs below or above in one tissue compared with the mean level of the 12 tissue types. Type II markers are genomic loci that demonstrate highly variable methylation densities across the 12 tissue types. A locus is considered highly variable when (A) the methylation density of the most hypermethylated tissue is at least 20% higher than that of the most hypomethylated one; and (B) the SD of the methylation densities across the 13 tissue types when divided by the mean methylation density (i.e., the coefficient of variation) of the group is at least 0.25. To reduce the number of potentially redundant markers, only one marker would be selected in one contiguous block of two CpG shores flanking one CGI.

### Example 4—Plasma cfDNA Tissue Deconvolution

The mathematical relationship between the methylation densities of the different methylation markers in plasma and the corresponding methylation markers in different tissues can be expressed as

$$\overline{{MD}_{ι}} = {\sum }_{k} {p}_{k} * M {D}_{i k}$$

where M̅D̅ᵢ̅ represents the methylation density of the methylation biomarker i in the plasma; pₖ represents the proportional contribution of tissue k to the plasma; and MDᵢₖ represents the methylation density of the methylation biomarker i in tissue k. The aim of the deconvolution process was to determine the proportional contribution of tissue k to the plasma, namely pₖ, for each member of the panel of tissues. Quadratic programming was used to solve the simultaneous equations. A matrix was compiled including the panel of tissues and their corresponding methylation densities for each methylation marker on the combined list of type I and type II markers (a total of 5,653 markers). The program input a range of pₖ values for each tissue type and determined the expected plasma DNA methylation density for each marker. The tested range of pₖ values should fulfill the expectation that the total contribution of all candidate tissues, namely, the liver, neutrophils, and lymphocytes, to plasma DNA would be 100% and the values of all pₖ would be nonnegative. These three tissue types were selected as each of them could be validated by one or more clinical scenarios, i.e. the liver in liver transplantation and HCC, and blood cells in bone marrow transplantation and the lymphoma case. The program then identified the set of pₖ values that resulted in expected methylation densities across the markers that most closely resembled the data obtained from the plasma DNA bisulfite sequencing.

The total contribution from T cells and B cells was regarded as the contribution from the lymphocytes, and the total contribution from white blood cells was regarded as the contribution from the lymphocytes and neutrophils.

### Example 5—Generating a Sequencing Library

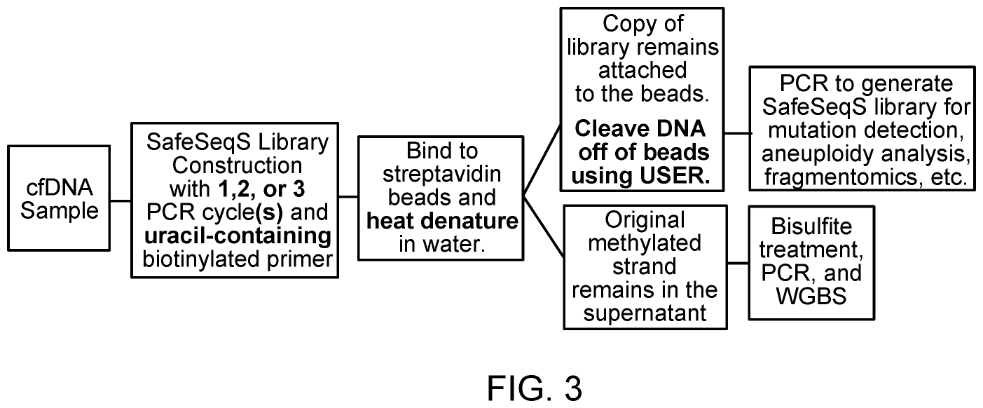

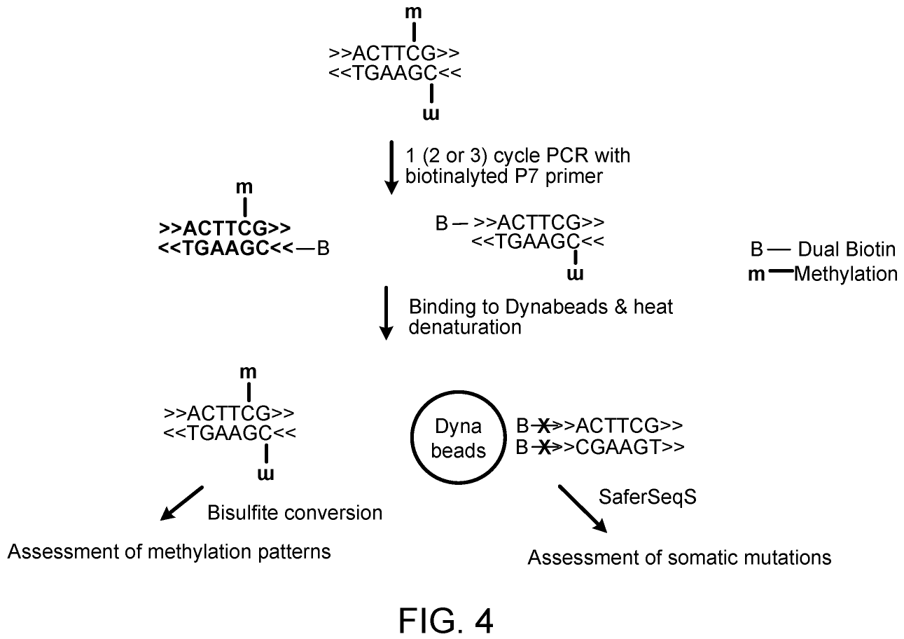

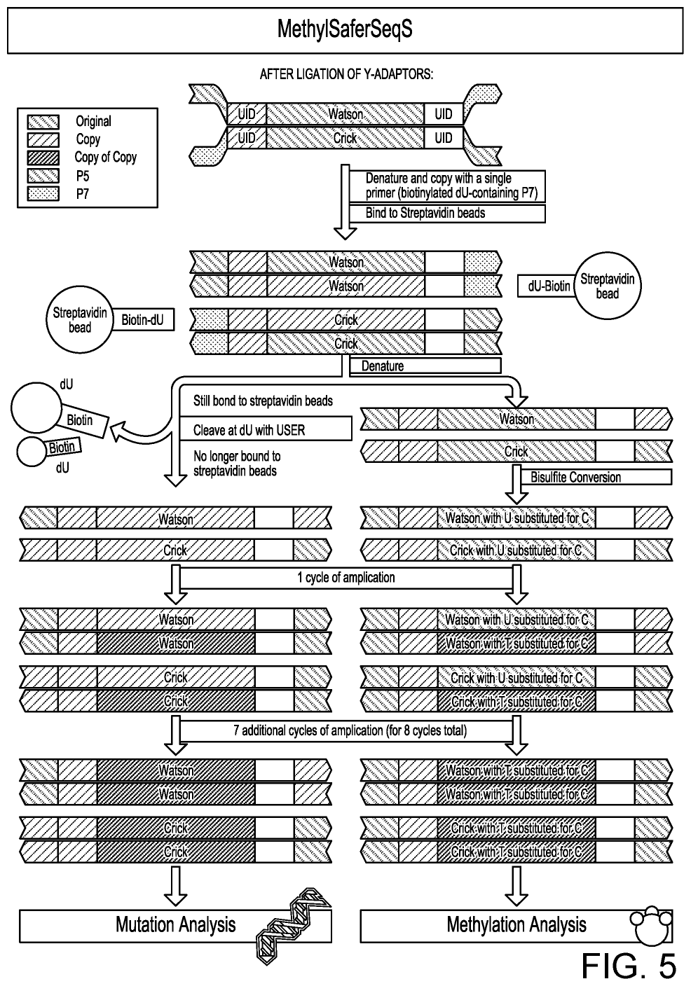

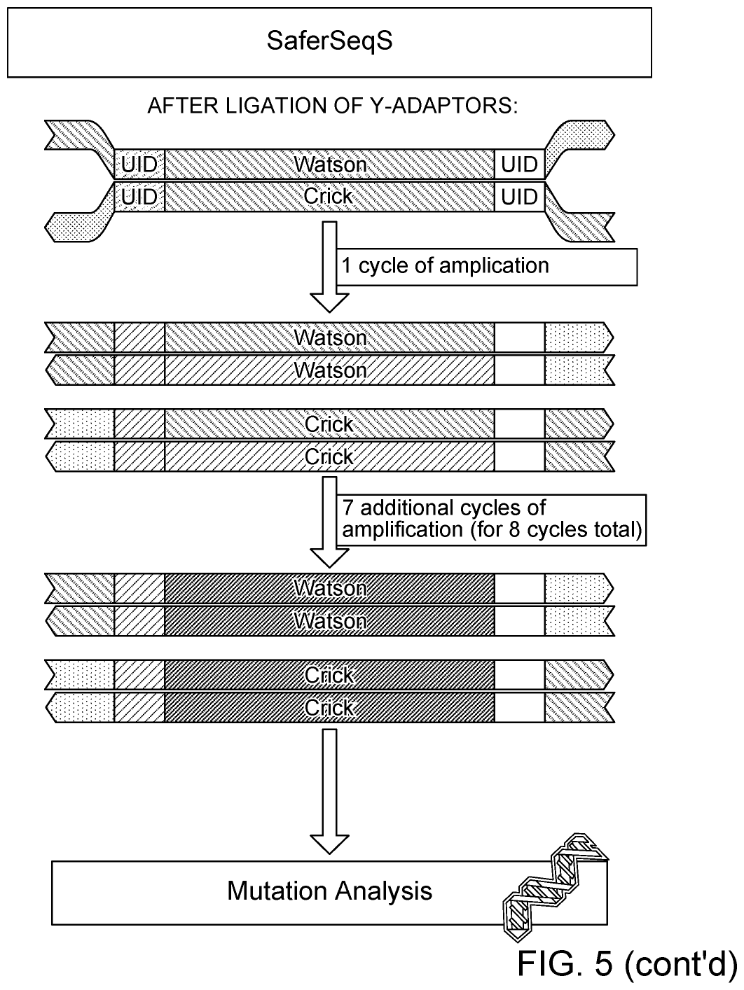

Libraries were prepared as described herein (FIG. 3). Custom 3′ and 5′ adaptors containing 5-methylcytosines rather than unmethylated cytosines were used during ligation, and following the ligations, one, two or three cycle(s) of linear PCR with a single, deoxyuridine-containing, biotinylated primer targeting the 3′ adaptor were performed. The amplified products were bound to streptavidin beads, and subsequently denatured to separate amplified strands (“A” pool) that were biotinylated from original, non-biotinylated strands that still preserved the methylation pattern (“M” pool). The supernatant following heat denaturation containing the “M” pool underwent bisulfite conversion and amplification. Following capture- or PCR-based enrichment, and/or whole genome-bisulfite sequencing, the “M” pool was analyzed for methylation changes. For the concurrent preparation of the “A” pool, the strands bound to streptavidin beads were released after treatment with the USER (Uracil-Specific Excision Reagent) enzyme, consisting of a mixture of Uracil DNA glycosylase and the DNA glycosylase-lyase Endonuclease VIII targeting the deoxyuridine base embedded within the 5′ ends of the strands. The released strands are amplified and sequenced for analysis of somatic mutations (e.g., Cohen et al. Nat Biotechnol. (2021) 39 (10): 1220-1227, which publication is hereby incorporated by reference) (FIG. 4-5).

What is claimed is:

1. A method for identifying a genetic characteristic and an epigenetic characteristic of a double-stranded DNA molecule in a population of double-stranded DNA molecules by assaying at least one strand of the double-stranded DNA molecule, the method comprising:
(a) attaching an adapter fragment to each end of the double-stranded DNA molecule to generate an adapted double-stranded DNA molecule, wherein the adapted double-stranded DNA molecule comprises an adapted Watson strand and an adapted Crick strand, wherein the adapter fragment comprises a molecular barcode, a primer sequence, and an adapter sequence, and wherein the molecular barcode of the adapted Watson strand is the reverse complement of the molecular barcode of the adapted Crick strand;
(b) copying both strands of the adapted double-stranded DNA molecule, wherein the copying comprises (i) contacting the adapted double-stranded DNA molecule with a tagged primer and (ii) performing a round of linear extension of the adapted double-stranded DNA molecule, generating a tagged Watson strand and a tagged Crick strand;
(c) subjecting the amplified products to denaturing conditions;
(d) separately recovering the adapted Watson and Crick strands and the tagged Watson and Crick strands;
(e) generating a first population of analyte DNA fragments from the tagged Watson and Crick strands and generating a first sequencing read for at least one member of the first population of analyte DNA fragments;
(f) generating a second population of analyte DNA fragments from the adapted Watson and Crick strands and generating a second sequencing read for at least one member of the second population of analyte DNA fragments;
(g) grouping the first sequencing reads according to the molecular barcode present on the at least one member of the first population of analyte DNA fragments to generate a first analyte DNA family;
(h) grouping the second sequencing reads according to the molecular barcode present on the at least one member of the second population of analyte DNA fragments to generate a second analyte DNA family;
(i) identifying the genetic characteristic of the tagged Watson and Crick strands in the first analyte DNA family; and
(j) identifying the epigenetic characteristic of the adapted Watson and Crick strands in the second analyte DNA family, thus, identifying the genetic characteristic and the epigenetic characteristic present on at least one strand of the double stranded DNA molecule.

2. The method of claim 1, wherein the adaptor fragment further comprises a sample barcode.

3. The method of claim 1 or 2, wherein the molecular barcode comprises an endogenous barcode, an exogenous barcode, or both.

4. The method of any one of claims 1-3, wherein the copying step (b) comprises performing one, two, or three round(s) of linear extension of the adapted double-stranded DNA molecule.

5. The method of any one of claims 1-4, wherein the tagged primer is a uracil-containing biotinylated primer, and wherein the tagged Watson and Crick strands are generated from the uracil-containing biotinylated primer.

6. The method of claim 5, wherein the recovering step (d) comprises contacting the tagged Watson and Crick strands with streptavidin-functionalized beads, and wherein the tagged Watson and Crick strands bind the streptavidin-functionalized beads.

7. The method of claim 6, wherein the recovered adapted Watson and Crick strands that are not bound to the streptavidin-functionalized beads are treated with bisulfite to convert Cytosine bases to Uracil bases to generate the second population of analyte DNA fragments comprising a population of converted DNA molecules.

8. The method of any one of claims 1-7, wherein the denaturing conditions comprise NaOH denaturation.

9. The method of any one of claims 1-8, wherein the denaturing conditions comprise heat denaturation, chemical denaturation, or combinations thereof.

10. The method of any one of claims 1-9, wherein the generating steps (e) and (f) are performed under PCR conditions.

11. The method of any one of claims 1-10, wherein the genetic characteristic is a mutation.

12. The method of claim 11, wherein the mutation is selected from the group consisting of an insertion, a deletion, a substitution, a deletion-insertion, a duplication, an inversion, a frameshift, a repeat expansion, a translocation, and combinations thereof.

13. The method of any one of claims 1-12, wherein the epigenetic characteristic is methylation.

14. The method of claim 13, wherein the epigenetic characteristic is a methylation pattern.

15. The method of claim 14, wherein the methylation pattern corresponds to a methylation pattern present in cells generated via clonal hematopoiesis of indeterminate origin.

16. The method of claim 15, wherein the methylation pattern corresponds to a methylation pattern present in a tissue of origin.

17. The method of claim 16, wherein the tissue of origin is the anus, bladder/urothelial, breast, cervix, colon/rectum, head and neck, kidney, liver/bile duct, lung, lymphoid neoplasm, melanoma, myeloid neoplasm, ovary, pancreas/gallbladder, prostate, thyroid, upper GI, or uterus.

18. The method of any one of claims 1-12, wherein the epigenetic characteristic is hydroxymethylation, histone modification, microRNA regulation, acetylation, phosphorylation, ubiquitination, or sumoylation.

19. The method of any one of claims 1-18, wherein the method identifies a genetic characteristic and an epigenetic characteristic of a double-stranded DNA molecule in a population of double-stranded DNA molecules by assaying both strands of the double-stranded DNA molecule.

20. A method for identifying a first characteristic and a second characteristic of a double stranded DNA molecule in a population of double-stranded DNA molecules by assaying at least one strand of the double-stranded DNA molecule, the method comprising:
(a) attaching an adapter fragment to each end of the double-stranded DNA molecule to generate an adapted double-stranded DNA molecule, wherein the adapted double-stranded DNA molecule comprises an adapted Watson strand and an adapted Crick strand, wherein the adapter fragment comprises a molecular barcode, a primer sequence, and an adapter sequence, and wherein the molecular barcode of the adapted Watson strand is the reverse complement of the molecular barcode of the adapted Crick strand;
(b) copying both strands of the adapted double-stranded DNA molecule, wherein the copying comprises (i) contacting the adapted double-stranded DNA molecule with a tagged primer and (ii) performing a round of linear extension of the adapted double-stranded DNA molecule, generating a tagged Watson strand and a tagged Crick strand;
(c) subjecting the amplified products to denaturing conditions;
(d) separately recovering the adapted Watson and Crick strands and the tagged Watson and Crick strands;
(e) generating a first population of analyte DNA fragments from the tagged Watson and Crick strands and generating a first sequencing read for at least one member of the first population of analyte DNA fragments;
(f) generating a second population of analyte DNA fragments from the adapted Watson and Crick strands and generating a second sequencing read for at least one member of the second population of analyte DNA fragments;
(g) grouping the first sequencing reads according to the molecular barcode present on the at least one member of the first population of analyte DNA fragments to generate a first analyte DNA family;
(h) grouping the second sequencing reads according to the molecular barcode present on the at least one member of the second population of analyte DNA fragments to generate a second analyte DNA family;
(i) identifying the first characteristic of the tagged Watson and Crick strands in the first analyte DNA family; and
(j) identifying the second characteristic of the adapted Watson and Crick strands in the second analyte DNA family, thus, identifying the first characteristic and the second characteristic present on at least one strand of the double-stranded DNA molecule.

21. The method of claim 20, wherein the adaptor fragment further comprises a sample barcode.

22. The method of claim 20 or 21, wherein the molecular barcode comprises an endogenous barcode, an exogenous barcode, or both

23. The method of any one of claims 20-22, wherein the copying step (b) comprises performing one, two, or three round(s) of linear extension of the adapted double-stranded DNA molecule.

24. The method of any one of claims 20-23, wherein the tagged primer is a uracil-containing biotinylated primer, and wherein the tagged Watson and Crick strands are generated from the uracil-containing biotinylated primer.

25. The method of claim 24, wherein the recovering step (d) comprises contacting the first single stranded DNA fragment with streptavidin-functionalized beads, and wherein the first single-stranded DNA fragment binds the streptavidin-functionalized beads.

26. The method of any one of claims 20-25, wherein the denaturing conditions comprise NaOH denaturation.

27. The method of any one of claims 20-26, wherein the denaturing conditions comprise heat denaturation, chemical denaturation, or combinations thereof.

28. The method of any one of claims 20-27, wherein the generating steps (e) and (f) are performed under PCR conditions.

29. The method of any one of claims 20-28, wherein the generating employs whole-genome PCR, whole-genome bisulfite sequencing, or capture sequencing.

30. The method of any one of claims 20-29, wherein the first characteristic is a genetic characteristic or an epigenetic characteristic.

31. The method of any one of claims 20-30, wherein the second characteristic is an epigenetic characteristic or an epigenetic characteristic.

32. The method of any one of claims 20-31, wherein the first characteristic and second characteristic are both genetic characteristics.

33. The method of any one of claims 20-31, wherein the first characteristic and second characteristic are both epigenetic characteristic.

34. The method of any one of claims 30-33, wherein the genetic characteristic is a mutation.

35. The method of claim 34, wherein the mutation is selected from the group consisting of an insertion, a deletion, a substitution, a deletion-insertion, a duplication, an inversion, a frameshift, a repeat expansion, a translocation, and combinations thereof.

36. The method of any one of claims 30-35, wherein identifying the genetic characteristic comprises mutational analysis, aneuploidy analysis, or fragmentomics.

37. The method of any one of claims 30-36, wherein the epigenetic characteristic is methylation.

38. The method of any one of claims 30-37, wherein the epigenetic characteristic is a methylation pattern.

39. The method of claim 38, wherein the methylation pattern corresponds to a methylation pattern present in cells generated via clonal hematopoiesis of indeterminate origin.

40. The method of claim 39, wherein the methylation pattern corresponds to a methylation pattern present in a tissue of origin.

41. The method of claim 40, wherein the tissue of origin is the anus, bladder/urothelial, breast, cervix, colon/rectum, head and neck, kidney, liver/bile duct, lung, lymphoid neoplasm, melanoma, myeloid neoplasm, ovary, pancreas/gallbladder, prostate, thyroid, upper GI, or uterus.

42. The method of any one of claims 30-41, wherein the epigenetic characteristic is hydroxymethylation, histone modification, microRNA regulation, acetylation, phosphorylation, ubiquitination, or sumoylation.

43. The method of any one of claims 20-42, wherein the method identifies a first characteristic and a second characteristic of a double stranded DNA molecule in a population of double-stranded DNA molecules by assaying both strands of the double-stranded DNA molecule.
